(19) United States
(12) Patent Application Publication
(10) Pub. No.: US 2025/0375450 A1
(43) Pub. Date: Dec. 11, 2025

(54) SMARCA2/4 INHIBITION AS A STRATEGY TO TREAT TUMORS THAT HARBOR ABERRANT BAF ASSEMBLIES

(71) Applicant: BOARD OF REGENTS, THE UNIVERSITY OF TEXAS SYSTEM, Austin, TX (US)

(72) Inventors: Yogesh K. Gupta, Helotes, TX (US); Stanto F. McHardy, San Antonio, TX (US)

(21) Appl. No.: 18/725,600

(22) PCT Filed: Jan. 5, 2023
(86) PCT No.: PCT/US2023/010236

§ 371 Date: Jun. 28, 2024

Related U.S. Application Data
(60) Provisional application No. 63/297,158, filed on Jan. 6, 2022.

(51) Int. Cl. A61K 31/517; A61P 35/00; C07D 239/95

(52) U.S. Cl. CPC A61K 31/517; A61P 35/00; C07D 239/95

(57) ABSTRACT

The present disclosure is concerned with substituted quinazoline-2,4-diamines and compositions for the treatment of disorders associated with altered expression of SMARCA2 and/or SMARCA4 such as, for example, cancer (e.g., sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, gliomas, leukemia, lymphoma, chronic myeloproliferative disorders, myelodysplastic syndrome, myeloproliferative neoplasm, non-small cell lung carcinoma, plasma cell neoplasm (myeloma)). This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/297,158, filed on Jan. 6, 2022, the contents of which are incorporated herein by reference in their entirety.

### BACKGROUND

The chromatin remodeler BAF (BRG1 or BRM-associated factors) complexes represent a combinatorial assembly of up to 15 different proteins including an essential SWI/SNF ATP motor subunit SMARCA4 or BRG1 or its biochemically redundant paralog SMARCA2 or BRM1. The SMARCA4/2-mediated chromatin remodeling activity promotes transcription factor recruitment, enhancer activation, and eviction of other BAF component(s) (St Pierre and Kadoch (2017) Curr Opin Genet Dev 42: 56-67). About 20% of all cancers harbor defective chromatin remodelers (Kadoch et al. (2013) Nat Genet 45: 592-601; Shain and Pollack (2013) PLoS One 8: e55119). As such, SMARCA4 mutations are frequent in tumors such as atypical teratoid/rhabdoid (AT/RT) (Hasselblatt et al. (2014) Acta Neuropathol 128: 453-456), lung (Imielinski et al. (2012) Cell 150: 1107-1120), medulloblastoma (Parsons et al. (2011) Science 331: 435-439), and small cell carcinoma of the ovary hypercalcemia type (SCCOHT) (Witkowski et al. (2014) Nat Genet 46: 438-443), which suggests that SMARCA4 may serve as a tumor suppressor gene. However, recent studies have linked the overexpression of SMARCA4 to oncogenesis and tumor maintenance in several tumor types, e.g., leukemia (Shi et al. (2013) Genes Dev 27: 2648-2662; Buscarlet et al. (2014) Blood 123: 1720-1728), TNBC (Wu et al. (2015) J Cell Physiol 230: 2683-2694; Wu et al. (2016) Oncotarget 7: 27158-27175), melanoma (Keenen et al. (2010) Oncogene 29: 81-92), and neuroblastoma (Jubierre et al. (2016) Oncogene 35: 5179-5190) (FIG. 1A and FIG. 1B, modified from Valencia, A. et al. (2019) Nat Cell Biol 2: 152-161).

The oncogenic role of BAF is also attributed to its perturbed function due to activity of residual BAF complexes (Helming et al. (2014) Cancer Cell 26: 309-317; Helming et al. (2014) Nat Med 20: 251-254). Thus, SMARCA2 has emerged as a synthetic lethal target in SMARCA4-deficient cancers (Hoffman et al. (2014) Proc Natl Acad Sci USA 111: 3128-3133; Wilson et al. (2014)Mol Cell Biol 34: 1136-1144). These tumors incorporate SMARCA2 to compensate for SMARCA4's loss of function as seen in non-small cell lung adenocarcinomas (NSCLCs) devoid of any alterations in known oncogenes such as EGFR and DDR2 (Hoffman et al. (2014) Proc Natl Acad Sci USA 111: 3128-3133; Wilson et al. (2014) Mol Cell Biol 34: 1136-1144; Oike et al. (2013) Cancer Res 73: 5508-5518). SMARCA2 depletion in these tumors suppressed the growth of tumor xenografts. Therefore, targeting the paralog dependency of residual/aberrant BAF complexes via small molecule inhibitors offers an attractive and novel approach to cancer treatment.

Moreover, recent studies have also clarified the oncogenic role of BAF complexes and molecular relationship of an EWSFLI1 and BAFs in Ewing's Sarcoma (EwS) (Boulay et al. (2017) Cell 171: 163-178 e119; Jayabal et al. (2021) Cell Rep 36: 109254). Molecular vulnerabilities that are based on synthetic lethality in cancers such as SWI/SNF deficiencies, may contribute to precision medicine. Of note, SMARCA2 does not seem to play a significant role in normal functioning of a cell as evident from fully viable SMARCA2 KO mice (Reyes et al. (1998) EMBO J 17: 6979-6991). Therefore, targeting SMARCA2 by small molecules should have minimal impact on normal cellular function. In addition, enhanced sensitivity to chemotherapy in knockdown of SMARCA4 in breast (Wu et al. (2016) Oncotarget 7: 27158-27175) and pancreatic (Davidson et al. (2018) Oncotarget 9: 9608-9617) cancer models provides a proof-of-principle for a SMARCA4/2 inhibitor to act as an effective adjuvant therapy. Despite the emergence of BAF inhibitors as a focus of drug discovery research (see, e.g., Papillon et al. (2018) J Med Chem 61: 10155-10172), there is no specific inhibitor of SMARCA2 available that could target its ATPase pocket or DNA binding surface. Thus, there remains a need for compounds and compositions for treating cancers having a tumor with a mutant BAF chromatin remodeling complex, and methods of making and using same.

### SUMMARY

In accordance with the purpose(s) of the invention, as embodied and broadly described herein, the invention, in one aspect, relates to substituted quinazoline-2,4-diamines and compositions for use in the prevention and treatment of disorders associated with altered expression of SMARCA2 and/or SMARCA4 such as, for example, cancers having a tumor with a mutant BAF chromatin remodeling complex (e.g., sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, gliomas, leukemia, lymphoma, chronic myeloproliferative disorders, myelodysplastic syndrome, myeloproliferative neoplasm, non-small cell lung carcinoma, plasma cell neoplasm (myeloma)).

Thus, disclosed are compounds having a structure represented by a formula:

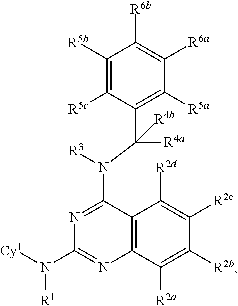

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R^{6a} is halogen; wherein R^{6b} is selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that either: (a) R^{6b} is chloro; (b) R^{6b} is hydrogen; or (c) R^{6b} is halogen and at least one of R^{5a}, R^{5b}, and R^{5c} is not hydrogen, or a pharmaceutically acceptable salt thereof.

Also disclosed are pharmaceutical compositions comprising a therapeutically effective amount of a disclosed compound, and a pharmaceutically acceptable carrier.

Also disclosed are methods of treating cancer in a subject in need thereof, the method comprising administering to the subject an effective amount of a compound having a structure represented by a formula:

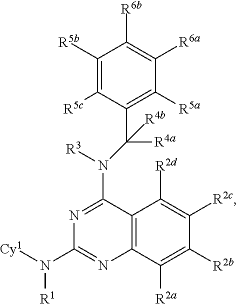

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{6a} and R^{6b} is independently selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof, wherein the cancer comprises a tumor having a mutant BAF chromatin remodeling complex.

Also disclosed are methods of modulating expression of one or more genes selected from SMARCA2 and SMARCA4 in a subject in need thereof, the method comprising administering to the subject a disclosed compound or a pharmaceutically acceptable salt thereof.

Also disclosed are methods of modulating expression of one or more genes selected from SMARCA2 and SMARCA4 in a cell, the method comprising contacting the cell with an effective amount of a disclosed compound or a pharmaceutically acceptable salt thereof.

Also disclosed are kits comprising a compound having a structure represented by a formula:

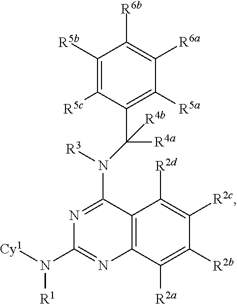

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{6a} and R^{6b} is independently selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof, and one or more selected from: (a) a chemotherapeutic agent; (b) instructions for administering the compound in connection with treating cancer; and (c) instructions for treating cancer.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings.

FIG. 1A shows representative schematics illustrating cancer associated genomic alterations in BAF subunits.

FIG. 1B shows representative data illustrating synthetic lethal relationships of BAFs that harbor defective subunits in cancer. For example, SMARCA4 deficient cancers rely upon SMARCA2 activity.

FIG. 2A shows a representative schematic illustrating the domain architecture of SMARCA2 (red bar, boundary of the most stable construct attempted for large scale protein expression and purification; aa 437-1493).

FIG. 2B shows representative Coomassie-stained SDS-PAGE gels showing the purity of the SMARCA2-1 protein.

FIG. 2C and FIG. 2D show representative data illustrating the ATPase activity of SMARCA2-1 in the presence of two DNA oligos (DNA_EcoP2 and DNA_EEPD1). The highest ATPase activity was observed with the DNA_EEPD1 duplex DNA.

FIG. 3A-F show representative dose response curves of compound nos. 7 (FIG. 3A) and 8 (FIG. 3B).

FIG. 4A shows representative data illustrating that expression of SMARCA2 in cancer cells was confirmed by western blotting. RMS, rhabdomyosarcoma.

FIG. 4B shows representative data illustrating the results of a 40,000-compound screen against human SMARCA2. A total of 14 compounds (red dots) were identified. Arrow indicates AT-301.

FIG. 4C shows the chemical structure of AT-301.

FIG. 4D shows representative data illustrating that one of the top hits, AT-301, inhibits the ATPase activity of SMARCA2 in a secondary ATPase assay with an IC₅₀ of ˜5 μM.

FIG. 4E shows representative data illustrating that AT-301 also attenuates the growth of the majority of Ewing's sarcoma cell lines tested, including ES1, ES6, and EW8. In vivo live imaging of ES3 cells in the presence of AT-301 (IC₅₀˜5 μM).

FIG. 5 shows representative data illustrating the change in % confluency of Ewing's sarcoma cells upon treatment with AT-301.

FIG. 6 shows representative data illustrating the change in % cell viability of Ewing's sarcoma cells post-treatment (72 hours) with AT-301.

FIG. 7A-C show representative data illustrating the change in % confluency of Rhabdomyosarcoma cells upon treatment with AT-301.

FIG. 8A-C show representative data illustrating the change in % cell viability of Rhabdomyosarcoma cells upon treatment with AT-301.

FIG. 9A-C show representative data illustrating that AT-301 attenuates the ATPase activity of SMARCA2 in different DNA/chromatin substrates. Specifically, FIG. 9A shows a representative SDS-page illustrating purified histone proteins that were used for reconstitution of nucleosome core particle (NCP). FIG. 9B shows representative data from an electromobility shift assay (EMSA) illustrating an ethidium bromide-stained 4-20% native PAGE gel showing that SMARCA2 forms a stable complex with reconstituted nucleosome core particle (NCP). Without wishing to be bound by, these data suggest that SMARCA2 forms a stable complex on in vitro reconstituted NCP. FIG. 9C shows representative data illustrating the ATPase activity of SMARCA2 as detected by an enzyme-coupled phosphate release assay at 360 nm wavelength. As shown, SMARCA2 is highly active on all three substrates tested, i.e., 147 bp nucleosomal DNA, 29 bp duplex DNA, and NCP. In the presence of 5 μM AT-301, the ATPase activity of SMARCA2 is uniformly reduced in reactions containing all three types of DNA substrates.

FIG. 10A-C show representative data illustrating that AT-301 kills Ewing's, RMS, GBM, AML, and other cancer cells. Cell lines were treated with serial dilutions (2×) of AT-301, and IC₅₀ values of each cell line were determined using PrestoBlue dye for cell viability following manufacturer's protocol (Invitrogen, A13261). See also Table 3.

FIG. 11A-D show representative data illustrating that AT-301 induces apoptosis and alters cell cycle. Referring to FIG. 11A and FIG. 11B, RH4 (FIG. 11A) and ES3 (FIG. 11B) cells were treated with the indicated concentrations of BAF-X-1 or DMSO in triplicate and apoptosis was detected using the IncuCyte Caspase 3/7 green dye (Sartorius). Brightfield and green fluorescence images were captured every 2 hours from 4 fields per well on the IncuCyte live cell imager. The proportion of caspase positive (green) cells relative to % confluence of the well was calculated on the IncuCyte software and plotted against time. Referring to FIG. 11C and FIG. 11D, RH41 cells were treated with 7.5 μM AT-301 (FIG. 11D) or DMSO (FIG. 11C) for 24 hours, harvested with accutase, washed, and fixed in 70% ethanol. They were subsequently stained with propidium iodide/RNase and fluorescence was analyzed on BD flow cytometer. Resulting data was analyzed using FlowJo software.

FIG. 12A-D show representative data illustrating that the assembly of BAF complex is perturbed by AT-301 inhibitor without perturbing oncogenic fusion protein levels. Referring to FIG. 12A and FIG. 12B, immunoblots of RH41 (FIG. 12A) and ES7 (FIG. 12B) cells after 4 and 12 hrs of treatment with AT-301 or DMSO. FOXO1 and FLI1 expression remains unchanged upon AT-301 treatment. Referring to FIG. 12C and FIG. 12D, gel filtration chromatography of ES3 (FIG. 12C) and RH41 (FIG. 12D) cell nuclear lysates after BAF-X-1 treatment are shown. Lysates were passed through a Sephacryl S-400 HR column and every fourth fraction was immuno-blotted for the proteins indicated with fraction numbers above the lanes. The immunoblots probed for different subunits of BAF show that BAF-X-1 perturbs the BAF complex assembly.

FIG. 13A and FIG. 13B show representative data illustrating that AT-301 suppresses tumor growth of Ewing's sarcoma in a patient-derived xenograft (PDX) model.

FIG. 14A and FIG. 14B show representative data illustrating that AT-301 inhibits the growth of ovarian (FIG. 14A) and endometrial (FIG. 14B) cancer cells in a dose-dependent manner.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀,” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease, disorder, or condition. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the condition being treated and the severity of the condition; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the 11 clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula-NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide” as used herein can be used interchangeably and refer to F, Cl, Br, or I.

The terms “pseudohalide,” “pseudohalogen,” or “pseudohalo” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A'S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄Rº; —(CH₂)₀₋₄ORº; —O(CH₂)₀₋₄Rº, —O—(CH₂)₀₋₄C(O)ORº; —(CH₂)₀₋₄CH(ORº)₂; —(CH₂)₀₋₄SRº; —(CH₂)₀₋₄Ph, which may be substituted with Rº; —(CH₂)₀₋₄O(CH₂)₀₋₁Ph which may be substituted with Rº; —CH═CHPh, which may be substituted with Rº; —(CH₂)₀₋₄O(CH₂)₀₋₁-pyridyl which may be substituted with Rº; —NO₂; —CN; —N₃; —(CH₂)₀₋₄N(Rº)₂; —(CH₂)₀₋₄N(Rº)C(O)Rº; —N(Rº)C(S)Rº; —(CH₂)₀₋₄N(Rº)C(O)NRº₂; —N(Rº)C(S)NRº₂; —(CH₂)₀₋₄N(Rº)C(O)ORº; —N(Rº)N(Rº)C(O)Rº; —N(Rº)N(Rº)C(O)NRº₂; —N(Rº)N(Rº)C(O)ORº; —(CH₂)₀₋₄C(O)Rº; —C(S)Rº; —(CH₂)₀₋₄C(O)ORº; —(CH₂)₀₋₄C(O)SRº; —(CH₂)₀₋₄C(O)OSiRº₃; —(CH₂)₀₋₄OC(O)Rº; —OC(O)(CH₂)₀₋₄SR—, SC(S)SRº; —(CH₂)₀₋₄SC(O)Rº; —(CH₂)₀₋₄C(O)NRº₂; —C(S)NRº₂; —C(S)SRº; —(CH₂)₀₋₄OC(O)NRº₂; —C(O)N(ORº)Rº; —C(O)C(O)Rº; —C(O)CH₂C(O)Rº; —C(NORº)Rº; —(CH₂)₀₋₄SSRº; —(CH₂)₀₋₄S(O)₂Rº; —(CH₂)₀₋₄S(O)₂ORº; —(CH₂)₀₋₄OS(O)₂Rº; —S(O)₂NRº₂; —(CH₂)₀₋₄S(O)Rº; —N(Rº)S(O)₂NRº₂; —N(Rº)S(O)₂Rº; —N(ORº)Rº; —C(NH)NRº₂; —P(O)₂Rº; —P(O)Rº₂; —OP(O)Rº₂; —OP(O)(ORº)₂; SiRº₃; —(C₁₋₄ straight or branched alkylene)O—N(Rº)₂; or —(C₁₋₄ straight or branched alkylene)C(O)O—N(Rº)₂, wherein each Rº may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of Rº, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on Rº (or the ring formed by taking two independent occurrences of Rº together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of Rº include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

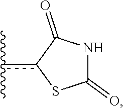

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di-, or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

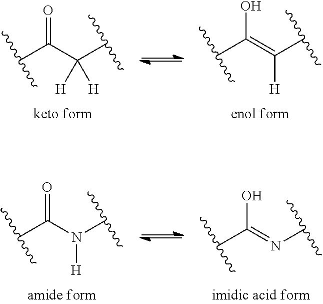 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

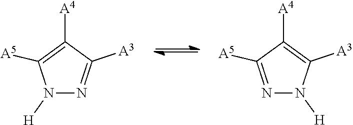

Unless stated to the contrary, the invention includes all such possible tautomers.

It is known that chemical substances form solids, which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

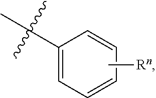

which is understood to be equivalent to a formula:

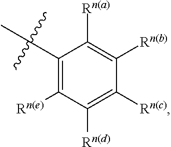

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, the invention relates to substituted quinazoline-2,4-diamines useful in preventing and treating a disorder associated with altered expression of SMARCA2 and/or SMARCA4. In a further aspect, the disorder is cancer. In a still further aspect, the cancer has a tumor with a mutant BAF chromatin remodeling complex (e.g., sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, gliomas, leukemia, lymphoma, chronic myeloproliferative disorders, myelodysplastic syndrome, myeloproliferative neoplasm, non-small cell lung carcinoma, plasma cell neoplasm (myeloma)).

In one aspect, the compounds of the invention are useful in the treatment of cancer, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

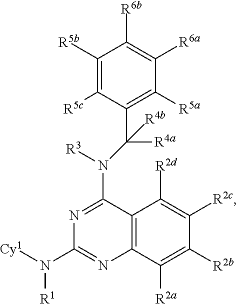

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R^{6a} is halogen; wherein R^{6b} is selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that either: (a) R^{6b} is chloro; (b) R^{6b} is hydrogen; or (c) R^{6b} is halogen and at least one of R^{5a}, R^{5b}, and R^{5c} is not hydrogen, or a pharmaceutically acceptable salt thereof.

In various aspects, the compound is present as a pharmaceutically acceptable salt. Examples of pharmaceutically acceptable salts include, but are not limited to, acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate, and xinofoate salts. In a further aspect, the compound is present as a hydrochloride salt.

In various aspects, the compound has a structure represented by a formula:

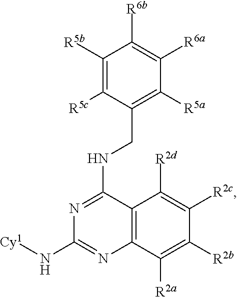

or a pharmaceutically acceptable salt thereof.

In various aspects, the compound has a structure represented by a formula:

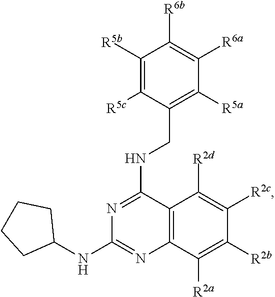

or a pharmaceutically acceptable salt thereof.

In various aspects, the compound has a structure represented by a formula:

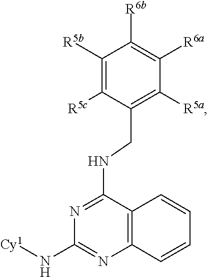

or a pharmaceutically acceptable salt thereof.

In various aspects, the compound has a structure represented by a formula:

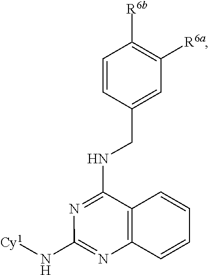

or a pharmaceutically acceptable salt thereof.

In various aspects, the compound has a structure represented by a formula:

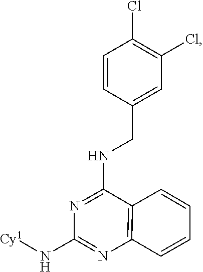

or a pharmaceutically acceptable salt thereof.

In various aspects, the compound is a structure selected from:

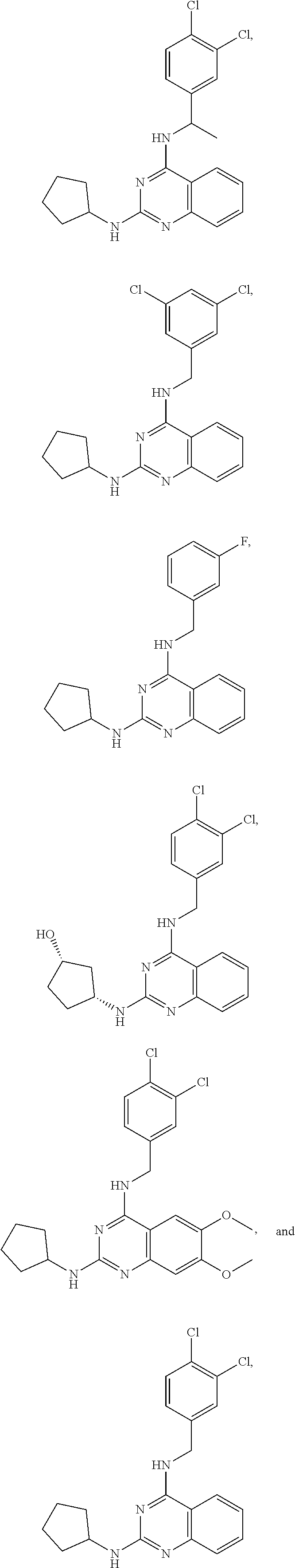 [SMILES: CC(Nc1nc(NC2CCCC2)nc2ccccc12)c1ccc(Cl)c(Cl)c1 ; Clc1cc(Cl)cc(CNc2nc(NC3CCCC3)nc3ccccc23)c1 ; Fc1cccc(CNc2nc(NC3CCCC3)nc3ccccc23)c1 ; O[C@H]1CC[C@@H](Nc2nc(NCc3ccc(Cl)c(Cl)c3)c3ccccc3n2)C1 ; COc1cc2nc(NC3CCCC3)nc(NCc3ccc(Cl)c(Cl)c3)c2cc1OC ; Clc1ccc(CNc2nc(NC3CCCC3)nc3ccccc23)cc1Cl]

or a pharmaceutically acceptable salt thereof.

In various aspects, the compound is:

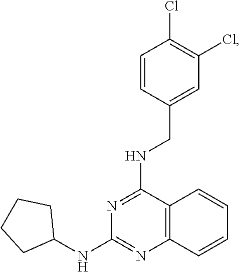 [SMILES: Clc1ccc(CNc2nc(NC3CCCC3)nc3ccccc23)cc1Cl]

or a pharmaceutically acceptable salt thereof.
a. R¹ Groups

In one aspect, R¹ is selected from hydrogen and C1-C4 alkyl. In a further aspect, R¹ is selected from hydrogen, methyl, ethyl, n-propyl, and isopropyl. In a still further aspect, R¹ is selected from hydrogen, methyl, and ethyl. In yet a further aspect, R¹ is selected from hydrogen and ethyl. In an even further aspect, R¹ is selected from hydrogen and methyl.

In various aspects, R¹ is C1-C4 alkyl. In a further aspect, R¹ is selected from methyl, ethyl, n-propyl, and isopropyl. In a still further aspect, R¹ is selected from methyl and ethyl. In yet a further aspect, R¹ is ethyl. In an even further aspect, R¹ is methyl.

In various aspects, R¹ is hydrogen.

b. R^{2a}, R^{2b}, R^{2c}, R^{2d}, and R^{2e} Groups

In one aspect, each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{2a}, R^{2b}, R^{2c}, R^{2d}, and R^{2e} is independently selected from hydrogen, —F, —Cl, —CN, —NH₂, —OH, —NO₂, methyl, ethyl, n-propyl, isopropyl, ethenyl, propenyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, R^{2d}, and R^{2e} is independently selected from hydrogen, —F, —Cl, —CN, —NH₂, —OH, —NO₂, methyl, ethyl, ethenyl, —CCl₃, —CF₃, —CHCl₂, —CHF₂, —CH₂Cl, —CH₂F, —CH₂CH₂Cl, —CH₂CH₂F, —CH₂CN, —CH₂CH₂CN, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{2a}, R^{2b}, R^{2c}, R^{2d}, and R^{2e} is independently selected from hydrogen, —F, —Cl, —CN, —NH₂, —OH, —NO₂, methyl, —CCl₃, —CF₃, —CHCl₂, —CHF₂, —CH₂Cl, —CH₂F, —CH₂CN, —CH₂OH, —OCCl₃, —OCF₃, —OCHCl₂, —OCHF₂, —OCH₂Cl, —OCH₂F, —OCH₃, —NHCH₃, —N(CH₃)₂, and —CH₂NH₂.

In various aspects, each of R^{2a}, R^{2b}, R^{2c}, R^{2d}, and R^{2e} is independently selected from hydrogen, halogen, —CN, —NH₂, —OH, —NO₂, C1-C4 alkyl, and C2-C4 alkenyl. In a further aspect, each of R^{2a}, R^{2b}, R^{2c}, R^{2d}, and R^{2e} is independently selected from hydrogen, —F, —Cl, —CN, —NH₂, —OH, —NO₂, methyl, ethyl, n-propyl, isopropyl, ethenyl, and propenyl. In a still further aspect, each R^{2a}, R^{2b}, R^{2c}, R^{2d}, and R^{2e} is independently selected from hydrogen, —F, —Cl, —CN, —NH₂, —OH, —NO₂, methyl, ethyl, and ethenyl. In yet a further aspect, each of R^{2a}, R^{2b}, R^{2c}, R^{2d}, and R^{2e} is independently selected from hydrogen, —F, —Cl, —CN, —NH₂, —OH, —NO₂, and methyl.

In various aspects, each of R^{2a}, R^{2b}, R^{2c}, R^{2d}, and R^{2e} is independently selected from hydrogen, C1-C4 alkyl, and C2-C4 alkenyl. In a further aspect, each of R^{2a}, R^{2b}, R^{2c}, R^{2d}, and R^{2e} is independently selected from hydrogen, methyl, ethyl, n-propyl, isopropyl, ethenyl, and propenyl. In a still further aspect, each of R^{2a}, R^{2b}, R^{2c}, R^{2d}, and R^{2e} is independently selected from hydrogen, methyl, ethyl, and ethenyl. In yet a further aspect, each of R^{2a}, R^{2b}, R^{2c}, R^{2d}, and R^{2e} is independently selected from hydrogen and methyl.

In various aspects, each of R^{2a}, R^{2b}, R^{2c}, R^{2d}, and R^{2e} is independently selected from hydrogen, halogen, —CN, —NH₂, —OH, —NO₂, C1-C4 haloalkyl, and C1-C4 cyanoalkyl. In a further aspect, each of R^{2a}, R^{2b}, R^{2c}, R^{2d}, and R^{2e} is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{2a}, R^{2b}, R^{2c}, R^{2d}, and R^{2e} is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{2a}, R^{2b}, R^{2c}, R^{2d}, and R^{2e} is independently selected from hydrogen, —F, —Cl, —CN, —NH₂, —OH, —NO₂, —CCl₃, —CF₃, —CHCl₂, —CHF₂, —CH₂Cl, —CH₂F, and —CH₂CN.

In various aspects, each of R^{2a}, R^{2b}, R^{2c}, R^{2d}, and R^{2e} is independently selected from hydrogen, C1-C4 haloalkyl, and C1-C4 cyanoalkyl. In a further aspect, each of R^{2a}, R^{2b}, R^{2c}, R^{2d}, and R^{2e} is independently selected from hydrogen, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{2a}, R^{2b}, R^{2c}, R^{2d}, and R^{2e} is independently selected from hydrogen, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{2a}, R^{2b}, R^{2c}, R^{2d}, and R^{2e} is independently selected from hydrogen, —CCl₃, —CF₃, —CHCl₂, —CHF₂, —CH₂Cl, —CH₂F, and —CH₂CN.

In various aspects, each of R^{2a}, R^{2b}, R^{2c}, R^{2d}, and R^{2e} is independently selected from hydrogen, halogen, —CN, —NH₂, —OH, —NO₂, C1-C4 hydroxyalkyl, C1-C4 haloalkoxy, and C1-C4 alkoxy. In a further aspect, each of R^{2a}, R^{2b}, R^{2c}, R^{2d}, and R^{2e} is independently selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{2a}, R^{2b}, R^{2c}, R^{2d}, and R^{2e} is independently selected from hydrogen, —F, —Cl, —CN, —NH₂, —OH, —NO₂, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{2a}, R^{2b}, R^{2c}, R^{2d}, and R^{2e} is independently selected from hydrogen, —F, —Cl, —CN, —NH₂, —OH, —NO₂, —CH₂OH, —OCCl₃, —OCF₃, —OCHCl₂, —OCHF₂, —OCH₂Cl, —OCH₂F, and —OCH₃.

In various aspects, each of R^{2a}, R^{2b}, R^{2c}, R^{2d}, and R^{2e} is independently selected from hydrogen, C1-C4 hydroxyalkyl, C1-C4 haloalkoxy, and C1-C4 alkoxy. In a further aspect, each of R^{2a}, R^{2b}, R^{2c}, R^{2d}, and R^{2e} is independently selected from hydrogen, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{2a}, R^{2b}, R^{2c}, R^{2d}, and R^{2e} is independently selected from hydrogen, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{2a}, R^{2b}, R^{2c}, R^{2d}, and R^{2e} is independently selected from hydrogen, —CH₂OH, —OCCl₃, —OCF₃, —OCHCl₂, —OCHF₂, —OCH₂Cl, —OCH₂F, and —OCH₃.

In various aspects, each of R^{2a}, R^{2b}, R^{2c}, R^{2d}, and R^{2e} is independently selected from hydrogen, halogen, —CN, —NH₂, —OH, —NO₂, C1-C4 alkylamino, (C1-C4)(C1-C4) dialkylamino, and C1-C4 aminoalkyl. In a further aspect, each of R^{2a}, R^{2b}, R^{2c}, R^{2d}, and R^{2e} is independently selected from hydrogen, —F, —Cl, —CN, —NH₂, —OH, —NO₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, R^{2d}, and R^{2e} is independently selected from hydrogen, —F, —Cl, —CN, —NH₂, —OH, —NO₂, —NHCH₃, —NHCH₂CH₃, —N(CH₃)₂, —N(CH₃)CH₂CH₃, —CH₂NH₂, —CH₂CH₂NH₂. In yet a further aspect, each of R^{2a}, R^{2b}, R^{2c}, R^{2d}, and R^{2e} is independently selected from hydrogen, —F, —Cl, —CN, —NH₂, —OH, —NO₂, —NHCH₃, —N(CH₃)₂, and —CH₂NH₂.

In various aspects, each of R^{2a}, R^{2b}, R^{2c}, R^{2d}, and R^{2e} is independently selected from hydrogen, C1-C4 alkylamino, (C1-C4)(C1-C4) dialkylamino, and C1-C4 aminoalkyl. In a further aspect, each of R^{2a}, R^{2b}, R^{2c}, R^{2d}, and R^{2e} is independently selected from hydrogen, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, R^{2d}, and R^{2e} is independently selected from hydrogen, —NHCH₃, —NHCH₂CH₃, —N(CH₃)₂, —N(CH₃)CH₂CH₃, —CH₂NH₂, and —CH₂CH₂NH₂. In yet a further aspect, each of R^{2a}, R^{2b}, R^{2c}, R^{2d}, and R^{2e} is independently selected from hydrogen, —NHCH₃, —N(CH₃)₂, and —CH₂NH₂.

In various aspects, each of R^{2a}, R^{2b}, R^{2c}, R^{2d}, and R^{2e} is independently selected from hydrogen, halogen, C1-C4 alkyl, C1-C4 haloalkyl, C1-C4 haloalkoxy, and C1-C4 alkoxy. In a further aspect, each of R^{2a}, R^{2b}, R^{2c}, R^{2d}, and R^{2e} is independently selected from hydrogen, —F, —Cl, methyl, ethyl, n-propyl, isopropyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{2a}, R^{2b}, R^{2c}, R^{2d}, and R^{2e} is independently selected from hydrogen, —F, —Cl, methyl, ethyl, —CCl₃, —CF₃, —CHCl₂, —CHF₂, —CH₂Cl, —CH₂F, —CH₂CH₂Cl, —CH₂CH₂F, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{2a}, R^{2b}, R^{2c}, R^{2d}, and R^{2e} is independently selected from hydrogen, —F, —Cl, methyl, —CCl₃, —CF₃, —CHCl₂, —CHF₂, —CH₂Cl, —CH₂F, —OCCl₃, —OCF₃, —OCHCl₂, —OCHF₂, —OCH₂Cl, —OCH₂F, and —OCH₃.

In various aspects, each of R^{2a}, R^{2b}, R^{2c}, R^{2d}, and R^{2e} is independently selected from hydrogen and halogen. In a further aspect, each of R^{2a}, R^{2b}, R^{2c}, R^{2d}, and R^{2e} is independently selected from hydrogen, —F, —Cl, and —Br. In a still further aspect, each of R^{2a}, R^{2b}, R^{2c}, R^{2d}, and R^{2e} is independently selected from hydrogen, —F, and —Cl. In yet a further aspect, each of R^{2a}, R^{2b}, R^{2c}, R^{2d}, and R^{2e} is independently selected from hydrogen and —F. In an even further aspect, each of R^{2a}, R^{2b}, R^{2c}, R^{2d}, and R^{2e} is independently selected from hydrogen and —Cl.

In various aspects, each of R^{2a}, R^{2b}, R^{2c}, R^{2a}, and R^{2e} is hydrogen. In a further aspect, at least one of R^{2a}, R^{2b}, R^{2c}, R^{2d}, and R^{2e} is hydrogen. In a still further aspect, two of R^{2a}, R^{2b}, R^{2c}, R^{2a}, and R^{2e} is hydrogen. In yet further aspect, three of R^{2a}, R^{2b}, R^{2c}, R^{2a}, and R^{2e} is hydrogen. In an even further aspect, four of R^{2a}, R^{2b}, R^{2c}, R^{2d}, and R^{2e} is hydrogen.

c. R³ Groups

In one aspect, R³ is selected from hydrogen and C1-C4 alkyl. In a further aspect, R³ is selected from hydrogen, methyl, ethyl, n-propyl, and isopropyl. In a still further aspect, R³ is selected from hydrogen, methyl, and ethyl. In yet a further aspect, R³ is selected from hydrogen and ethyl. In an even further aspect, R³ is selected from hydrogen and methyl.

In various aspects, R³ is CT-C4 alkyl. In a further aspect, R³ is selected from methyl, ethyl, n-propyl, and isopropyl. In a still further aspect, R³ is selected from methyl and ethyl. In yet a further aspect, R³ is ethyl. In an even further aspect, R³ is methyl.

In various aspects, R³ is hydrogen.

d. R^{4a} and R^{4b} Groups

In one aspect, each of R^{4a} and R^{4b} is independently selected from hydrogen and C1-C4 alkyl. In a further aspect, each of R^{4a} and R^{4b} is independently selected from hydrogen, methyl, ethyl, n-propyl, and isopropyl. In a still further aspect, each of R^{4a} and R^{4b} is independently selected from hydrogen, methyl, and ethyl. In yet a further aspect, each of R^{4a} and R^{4b} is independently selected from hydrogen and ethyl. In an even further aspect, each of R^{4a} and R^{4b} is independently selected from hydrogen and methyl.

In various aspects, each of R^{4a} and R^{4b} is independently C1-C4 alkyl. In a further aspect, each of R^{4a} and R^{4b} is independently selected from methyl, ethyl, n-propyl, and isopropyl. In a still further aspect, each of R^{4a} and R^{4b} is independently selected from methyl and ethyl. In yet a further aspect, each of R^{4a} and R^{4b} is independently ethyl. In an even further aspect, each of R^{4a} and R^{4b} is independently methyl.

In various aspects, each of R^{4a} and R^{4b} is independently hydrogen.

e. R^{5a}, R^{5b}, and R^{5c} Groups

In one aspect, each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{5a}, R^{5b}, and R^{5c} is independently selected from hydrogen, —F, —Cl, —CN, —NH₂, —OH, —NO₂, methyl, ethyl, n-propyl, isopropyl, ethenyl, propenyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{5a}, R^{5b}, and R^{5c} is independently selected from hydrogen, —F, —Cl, —CN, —NH₂, —OH, —NO₂, methyl, ethyl, ethenyl, —CCl₃, —CF₃, —CHCl₂, —CHF₂, —CH₂Cl, —CH₂F, —CH₂CH₂Cl, —CH₂CH₂F, —CH₂CN, —CH₂CH₂CN, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{5a}, R^{5b}, and R^{5c} is independently selected from hydrogen, —F, —Cl, —CN, —NH₂, —OH, —NO₂, methyl, —CCl₃, —CF₃, —CHCl₂, —CHF₂, —CH₂Cl, —CH₂F, —CH₂CN, —CH₂OH, —OCCl₃, —OCF₃, —OCHCl₂, —OCHF₂, —OCH₂Cl, —OCH₂F, —OCH₃, —NHCH₃, —N(CH₃)₂, and —CH₂NH₂.

In various aspects, each of R^{5a}, R^{5b}, and R^{5c} is independently selected from hydrogen, halogen, —CN, —NH₂, —OH, —NO₂, C1-C4 alkyl, and C2-C4 alkenyl. In a further aspect, each of R^{5a}, R^{5b}, and R^{5c} is independently selected from hydrogen, —F, —Cl, —CN, —NH₂, —OH, —NO₂, methyl, ethyl, n-propyl, isopropyl, ethenyl, and propenyl. In a still further aspect, each R^{5a}, R^{5b}, and R^{5c} is independently selected from hydrogen, —F, —Cl, —CN, —NH₂, —OH, —NO₂, methyl, ethyl, and ethenyl. In yet a further aspect, each of R^{5a}, R^{5b}, and R^{5c} is independently selected from hydrogen, —F, —Cl, —CN, —NH₂, —OH, —NO₂, and methyl.

In various aspects, each of R^{5a}, R^{5b}, and R^{5c} is independently selected from hydrogen, C1-C4 alkyl, and C2-C4 alkenyl. In a further aspect, each of R^{5a}, R^{5b}, and R^{5c} is independently selected from hydrogen, methyl, ethyl, n-propyl, isopropyl, ethenyl, and propenyl. In a still further aspect, each of R^{5a}, R^{5b}, and R^{5c} is independently selected from hydrogen, methyl, ethyl, and ethenyl. In yet a further aspect, each of R^{5a}, R^{5b}, and R^{5c} is independently selected from hydrogen and methyl.

In various aspects, each of R^{5a}, R^{5b}, and R^{5c} is independently selected from hydrogen, halogen, —CN, —NH₂, —OH, —NO₂, C1-C4 haloalkyl, and C1-C4 cyanoalkyl. In a further aspect, each of R^{5a}, R^{5b}, and R^{5c} is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{5a}, R^{5b}, and R^{5c} is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{5a}, R^{5b}, and R^{5c} is independently selected from hydrogen, —F, —Cl, —CN, —NH₂, —OH, —NO₂, —CCl₃, —CF₃, —CHCl₂, —CHF₂, —CH₂Cl, —CH₂F, and —CH₂CN.

In various aspects, each of R^{5a}, R^{5b}, and R^{5c} is independently selected from hydrogen, C1-C4 haloalkyl, and C1-C4 cyanoalkyl. In a further aspect, each of R^{5a}, R^{5b}, and R^{5c} is independently selected from hydrogen, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{5a}, R^{5b}, and R^{5c} is independently selected from hydrogen, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{5a}, R^{5b}, and R^{5c} is independently selected from hydrogen, —CCl₃, —CF₃, —CHCl₂, —CHF₂, —CH₂Cl, —CH₂F, and —CH₂CN.

In various aspects, each of R^{5a}, R^{5b}, and R^{5c} is independently selected from hydrogen, halogen, —CN, —NH₂, —OH, —NO₂, C1-C4 hydroxyalkyl, C1-C4 haloalkoxy, and C1-C4 alkoxy. In a further aspect, each of R^{5a}, R^{5b}, and R^{5c} is independently selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{5a}, R^{5b}, and R^{5c} is independently selected from hydrogen, —F, —Cl, —CN, —NH₂, —OH, —NO₂, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{5a}, R^{5b}, and R^{5c} is independently selected from hydrogen, —F, —Cl, —CN, —NH₂, —OH, —NO₂, —CH₂OH, —OCCl₃, —OCF₃, —OCHCl₂, —OCHF₂, —OCH₂Cl, —OCH₂F, and —OCH₃.

In various aspects, each of R^{5a}, R^{5b}, and R^{5c} is independently selected from hydrogen, C1-C4 hydroxyalkyl, C1-C4 haloalkoxy, and C1-C4 alkoxy. In a further aspect, each of R^{5a}, R^{5b}, and R^{5c} is independently selected from hydrogen, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{5a}, R^{5b}, and R^{5c} is independently selected from hydrogen, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{5a}, R^{5b}, and R^{5c} is independently selected from hydrogen, —CH₂OH, —OCCl₃, —OCF₃, —OCHCl₂, —OCHF₂, —OCH₂Cl, —OCH₂F, and —OCH₃.

In various aspects, each of R^{5a}, R^{5b}, and R^{5c} is independently selected from hydrogen, halogen, —CN, —NH₂, —OH, —NO₂, C1-C4 alkylamino, (C1-C4)(C1-C4) dialkylamino, and C1-C4 aminoalkyl. In a further aspect, each of R^{5a}, R^{5b}, and R^{5c} is independently selected from hydrogen, —F, —Cl, —CN, —NH₂, —OH, —NO₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{5a}, R^{5b}, and R^{5c} is independently selected from hydrogen, —F, —Cl, —CN, —NH₂, —OH, —NO₂, —NHCH₃, —NHCH₂CH₃, —N(CH₃)₂, —N(CH₃)CH₂CH₃, —CH₂NH₂, —CH₂CH₂NH₂. In yet a further aspect, each of R^{5a}, R^{5b}, and R^{5c} is independently selected from hydrogen, —F, —Cl, —CN, —NH₂, —OH, —NO₂, —NHCH₃, —N(CH₃)₂, and —CH₂NH₂.

In various aspects, each of R^{5a}, R^{5b}, and R^{5c} is independently selected from hydrogen, C1-C4 alkylamino, (C1-C4)(C1-C4) dialkylamino, and C1-C4 aminoalkyl. In a further aspect, each of R^{5a}, R^{5b}, and R^{5c} is independently selected from hydrogen, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{5a}, R^{5b}, and R^{5c} is independently selected from hydrogen, —NHCH₃, —NHCH₂CH₃, —N(CH₃)₂, —N(CH₃)CH₂CH₃, —CH₂NH₂, and —CH₂CH₂NH₂. In yet a further aspect, each of R^{5a}, R^{5b}, and R^{5c} is independently selected from hydrogen, —NHCH₃, —N(CH₃)₂, and —CH₂NH₂.

In various aspects, each of R^{5a}, R^{5b}, and R^{5c} is independently selected from hydrogen, halogen, CT-C4 alkyl, CT-C4 haloalkyl, CT-C4 haloalkoxy, and CT-C4 alkoxy. In a further aspect, each of R^{5a}, R^{5b}, and R^{5c} is independently selected from hydrogen, —F, —Cl, methyl, ethyl, n-propyl, isopropyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{5a}, R^{5b}, and R^{5c} is independently selected from hydrogen, —F, —Cl, methyl, ethyl, —CCl₃, —CF₃, —CHCl₂, —CHF₂, —CH₂Cl, —CH₂F, —CH₂CH₂Cl, —CH₂CH₂F, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{5a}, R^{5b}, and R^{5c} is independently selected from hydrogen, —F, —Cl, methyl, —CCl₃, —CF₃, —CHCl₂, —CHF₂, —CH₂Cl, —CH₂F, —OCCl₃, —OCF₃, —OCHCl₂, —OCHF₂, —OCH₂Cl, —OCH₂F, and —OCH₃.

In various aspects, each of R^{5a}, R^{5b}, and R^{5c} is independently selected from hydrogen and halogen. In a further aspect, each of R^{5a}, R^{5b}, and R^{5c} is independently selected from hydrogen, —F, —Cl, and —Br. In a still further aspect, each of R^{5a}, R^{5b}, and R^{5c} is independently selected from hydrogen, —F, and —Cl. In yet a further aspect, each of R^{5a}, R^{5b}, and R^{5c} is independently selected from hydrogen and —F. In an even further aspect, each of R^{5a}, R^{5b}, and R^{5c} is independently selected from hydrogen and —Cl.

In various aspects, each of R^{5a}, R^{5b}, and R^{5c} is hydrogen. In a further aspect, at least one of R^{5a}, R^{5b}, and R^{5c} is hydrogen. In a still further aspect, two of R^{5a}, R^{5b}, and R^{5c} is hydrogen.

f. R^{6a} and R^{6b} Groups

In one aspect, R^{6a} is halogen and R^{6b} is selected from hydrogen and halogen.

In one aspect, each of R^{6a} and R^{6b} is independently selected from hydrogen and halogen. In a further aspect, each of R^{6a} and R^{6b} is independently selected from hydrogen, fluoro, chloro, and bromo. In a still further aspect, each of R^{6a} and R^{6b} is independently selected from hydrogen, fluoro, and chloro. In yet a further aspect, each of R^{6a} and R^{6b} is independently selected from hydrogen and fluoro. In an even further aspect, each of R^{6a} and R^{6b} is independently selected from hydrogen and chloro.

In various aspects, R^{6a} is halogen. In a further aspect, R^{6a} is selected from fluoro, chloro, and bromo. In a still further aspect, R^{6a} is selected from fluoro, chloro, and iodo. In yet a further aspect, R^{6a} is selected from fluoro, bromo, and iodo. In an even further aspect, R^{6a} is selected from chloro, bromo, and iodo. In a still further aspect, R^{6a} is selected from fluoro and chloro. In yet a further aspect, R^{6a} is selected from fluoro and bromo. In an even further aspect, R^{6a} is selected from fluoro and iodo. In a still further aspect, R^{6a} is selected from chloro and bromo. In yet a further aspect, R^{6a} is selected from chloro and iodo. In an even further aspect, R^{6a} is selected from bromo and iodo. In a still further aspect, R^{6a} is iodo. In yet a further aspect, R^{6a} is bromo. In an even further aspect, R^{6a} is chloro. In a still further aspect, R^{6a} is fluoro.

In various aspects, R^{6b} is selected from hydrogen and halogen. In a further aspect, R^{6b} is selected from hydrogen, fluoro, chloro, and bromo. In a still further aspect, R^{6b} is selected from hydrogen, fluoro, and chloro. In yet a further aspect, R^{6b} is selected from hydrogen and fluoro. In an even further aspect, R^{6b} is selected from hydrogen and chloro.

In various aspects, R^{6b} is halogen. In a further aspect, R^{6b} is selected from fluoro, chloro, and bromo. In a still further aspect, R^{6b} is selected from fluoro, chloro, and iodo. In yet a further aspect, R^{6b} is selected from fluoro, bromo, and iodo. In an even further aspect, R^{6b} is selected from chloro, bromo, and iodo. In a still further aspect, R^{6b} is selected from fluoro and chloro. In yet a further aspect, R^{6b} is selected from fluoro and bromo. In an even further aspect, R^{6b} is selected from fluoro and iodo. In a still further aspect, R^{6b} is selected from chloro and bromo. In yet a further aspect, R^{6b} is selected from chloro and iodo. In an even further aspect, R^{6b} is selected from bromo and iodo. In a still further aspect, R^{6b} is iodo. In yet a further aspect, R^{6b} is bromo. In an even further aspect, R^{6b} is chloro. In a still further aspect, R^{6b} is fluoro.

In various aspects, R^{6a} is hydrogen.

In various aspects, R^{6b} is hydrogen.

In one aspect, each of R^{6a} and R^{6b} is independently halogen. In a further aspect, each of R^{6a} and R^{6b} is independently selected from fluoro, chloro, and bromo. In a still further aspect, each of R^{6a} and R^{6b} is independently selected from fluoro and chloro. In yet a further aspect, each of R^{6a} and R^{6b} is fluoro. In an even further aspect, each of R^{6a} and R^{6b} is chloro.

g. Cy¹ Groups

In one aspect, Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹ is a C3-C8 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is a C3-C8 cycloalkyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹ is a C3-C8 cycloalkyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹ is a C3-C8 cycloalkyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is an unsubstituted C3-C8 cycloalkyl.

In various aspects, Cy¹ is a C3-C6 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹ is a C3-C6 cycloalkyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is a C3-C6 cycloalkyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹ is a C3-C6 cycloalkyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹ is a C3-C6 cycloalkyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is an unsubstituted C3-C6 cycloalkyl.

In various aspects, Cy¹ is a cyclopentyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Cy¹ is a cyclopentyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is a cyclopentyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Cy¹ is a cyclopentyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Cy¹ is a cyclopentyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Cy¹ is an unsubstituted cyclopentyl.

### 2. Example Compounds

In one aspect, a compound can be present as:

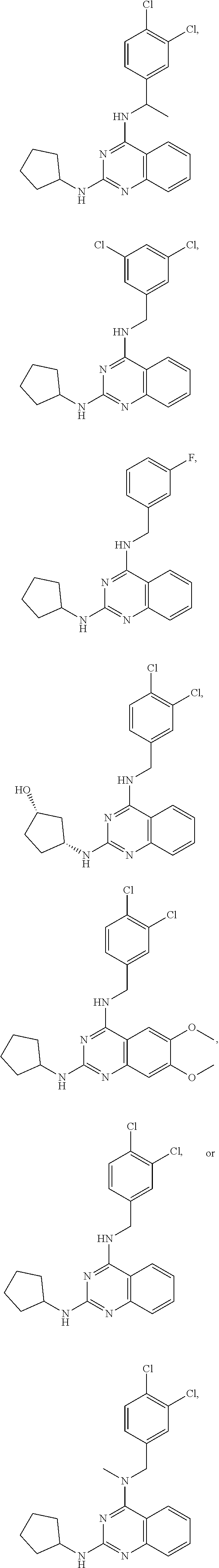

or a pharmaceutically acceptable salt thereof.

In one aspect, a compound can be present as:

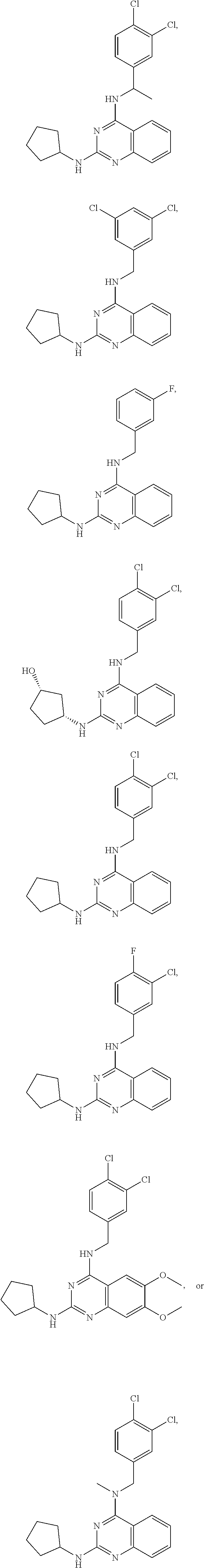

or a pharmaceutically acceptable salt thereof.

In one aspect, a compound can be present as:

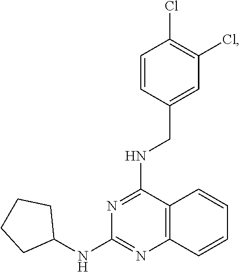 [SMILES: Clc1ccc(CNc2nc(NC3CCCC3)nc3ccccc23)cc1Cl]

or a pharmaceutically acceptable salt thereof.

It is contemplated that one or more compounds can optionally be omitted from the disclosed invention.

It is understood that the disclosed compounds can be used in connection with the disclosed methods, compositions, kits, and uses.

It is understood that pharmaceutical acceptable derivatives of the disclosed compounds can be used also in connection with the disclosed methods, compositions, kits, and uses. The pharmaceutical acceptable derivatives of the compounds can include any suitable derivative, such as pharmaceutically acceptable salts as discussed below, isomers, radiolabeled analogs, tautomers, and the like.

### C. PHARMACEUTICAL COMPOSITIONS

In one aspect, disclosed are pharmaceutical compositions comprising an effective amount of a disclosed compound and a pharmaceutically acceptable carrier.

Thus, in one aspect, disclosed are pharmaceutical compositions comprising an effective amount of a compound having a structure represented by a formula:

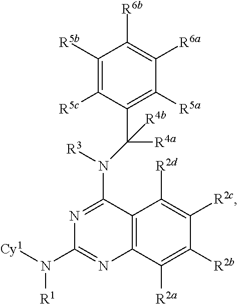

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R^{6a} is halogen; wherein R^{6b} is selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that either: (a) R^{6b} is chloro; (b) R^{6b} is hydrogen; or (c) R^{6b} is halogen and at least one of R^{5a}, R^{5b}, and R^{5c} is not hydrogen, or a pharmaceutically acceptable salt thereof, and a pharmaceutically acceptable carrier.

In various aspects, the compound is a structure selected from:

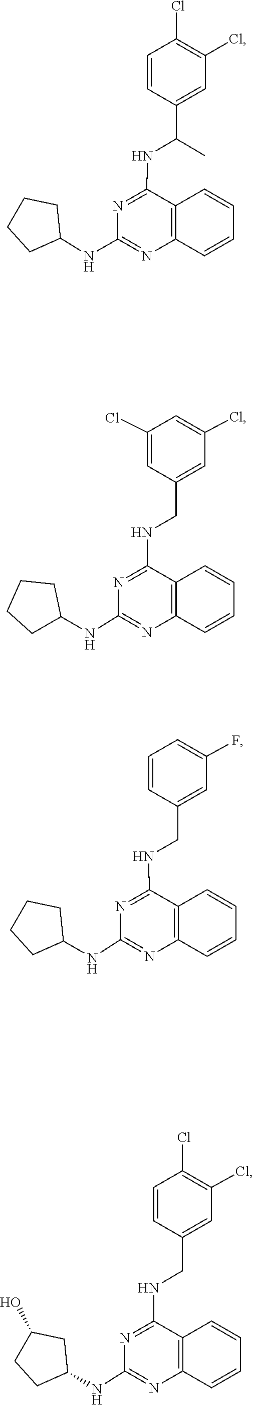 [SMILES: CC(Nc1nc(NC2CCCC2)nc2ccccc12)c1ccc(Cl)c(Cl)c1 ; Clc1cc(Cl)cc(CNc2nc(NC3CCCC3)nc3ccccc23)c1 ; Fc1cccc(CNc2nc(NC3CCCC3)nc3ccccc23)c1 ; O[C@H]1CC[C@@H](Nc2nc(NCc3ccc(Cl)c(Cl)c3)c3ccccc3n2)C1]

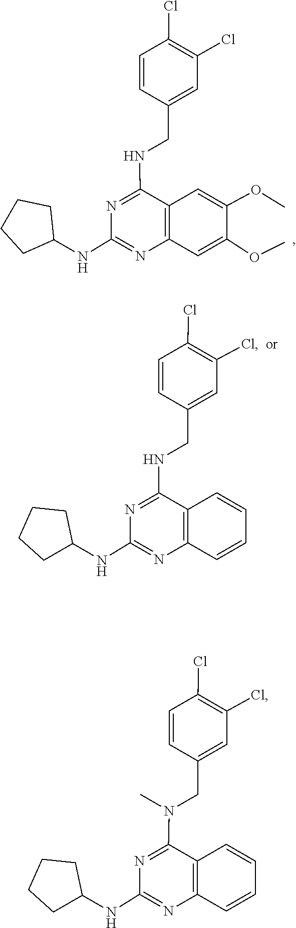

or a pharmaceutically acceptable salt thereof.

In various aspects, the compound is:

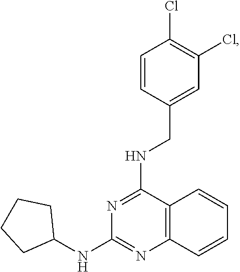 [SMILES: Clc1ccc(CNc2nc(NC3CCCC3)nc3ccccc23)cc1Cl]

or a pharmaceutically acceptable salt thereof.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, PA 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a disorder associated with altered expression of SMARCA2 and/or SMARCA4. In a still further aspect, the disorder is a cancer. In yet a further aspect, the cancer has a tumor with a mutant BAF chromatin remodeling complex (e.g., leukemias, neuroblastomas, melanomas, breast cancer, lung cancer, etc.).

It is understood that the disclosed compositions can be prepared from the disclosed compounds. It is also understood that the disclosed compositions can be employed in the disclosed methods of using.

### D. METHODS OF MAKING A COMPOUND

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Preferred methods include, but are not limited to, those described below. During any of the following synthetic sequences, it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, John Wiley & Sons, 1991, which are hereby incorporated by reference.

Reactions used to generate the compounds of this invention are prepared by employing reactions as shown in the following Reaction Schemes, as described and exemplified below. In certain specific examples, the disclosed compounds can be prepared by Route I, as described and exemplified below. The following examples are provided so that the invention might be more fully understood, are illustrative only, and should not be construed as limiting.

### 1. Route I

In one aspect, substituted quinazoline-2,4-diamines, or their pharmaceutically acceptable salts, can be prepared as shown below. Isolation and purification of the products is accomplished by standard procedures, which are known to a chemist of ordinary skill.

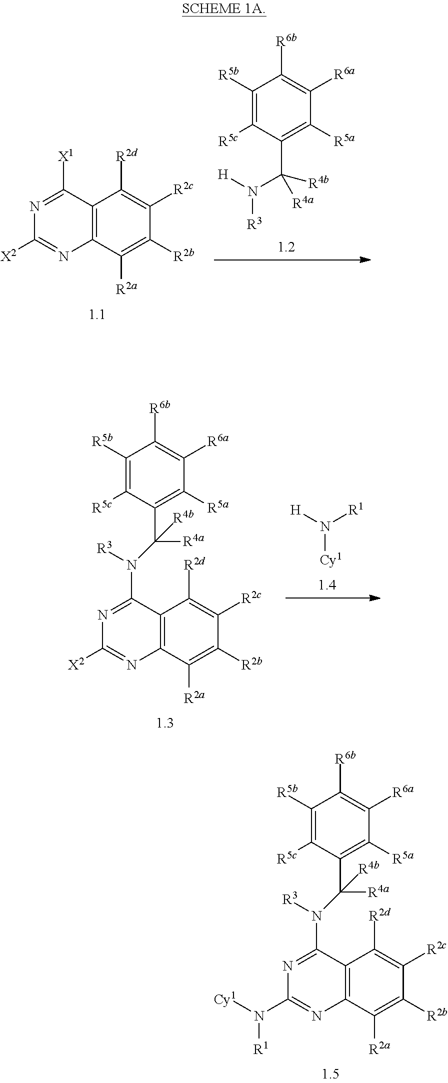

Compounds are represented in generic form, wherein X¹ and X² are independently halogen, and with substituents as noted in compound descriptions elsewhere herein. A more specific example is set forth below.

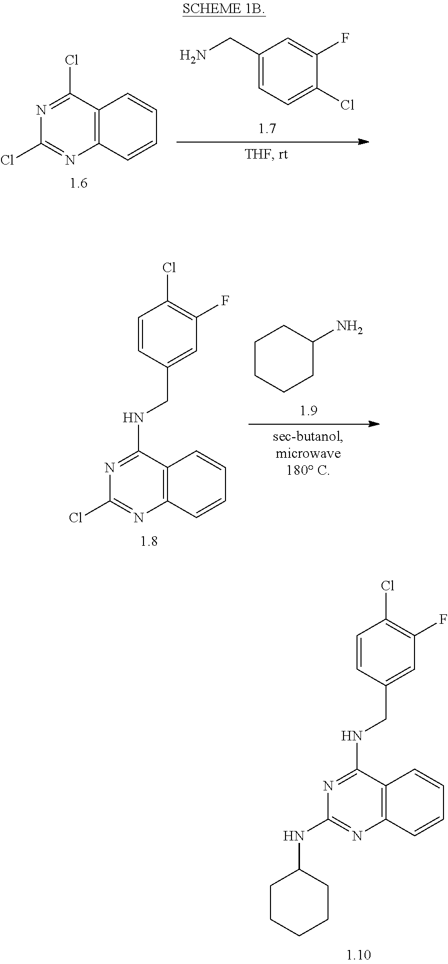 [SMILES: Clc1nc(Cl)c2ccccc2n1 ; NCc1ccc(Cl)c(F)c1 ; Fc1cc(CNc2nc(Cl)nc3ccccc23)ccc1Cl ; NC1CCCCC1 ; Fc1cc(CNc2nc(NC3CCCCC3)nc3ccccc23)ccc1Cl]

Referring to Scheme 1B above, condensation of an appropriate 2,4-dichloroquinazoline, e.g., 1.6 as shown above, with an appropriately substituted benzyl amine, e.g., 1.7 as shown above, in a suitable solvent such as THF, 2-methyl THF, DCE, or dioxane, at temperatures ranging from room temperature to 100° C., produces the desired mono-substituted aminoquinazoline, e.g., 1.8 as shown above. Appropriate quinazolines and appropriate benzyl amines are commercially available or prepared by methods known to one skilled in the art. Preferred conditions for this reaction include the use of THF as the solvent at room temperature. Treatment of an appropriate aminoquinazoline, e.g., 1.8 as shown above, with an appropriate cyclic amine, e.g., 1.9 as shown above, in a suitable alcoholic solvent such as sec-butanol, butanol, pentanol, ethanol, or t-butyl alcohol, under microwave irradiation conditions (120-180° C.), produces the desired quinazoline-2,4-diamine, e.g., 1.10 as shown above. Appropriate cyclic amines are commercially available or prepared by methods known to one skilled in the art. Preferred conditions for this reaction include the use of sec-butanol as the solvent under microwave irradiation conditions at a temperature of 180° C. As can be appreciated by one skilled in the art, the above reaction provides an example of a generalized approach wherein compounds similar in structure to the specific reactants above (compounds similar to compounds of type 1.1, 1.2, 1.3, and 1.4), can be substituted in the reaction to provide substituted quinazoline-2,4-diamine derivatives similar to Formula 1.5.

Pharmaceutically acceptable salts of the disclosed compounds include the acid or base addition salts thereof. All salt formation reactions are typically carried out in solution. The resulting salt may precipitate out and be collected by filtration or may be recovered by evaporation of the solvent. The degree of ionization in the resulting salt may vary from completely ionized to almost non-ionized. Suitable non-toxic, acid-addition pharmaceutically acceptable salts include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable non-toxic, base-addition pharmaceutically acceptable salts include, but are not limited to, the aluminum, arginine, benzathine, calcium, choline, diethylamine, diolamine, glycine, lysine, magnesium, meglumine, olamine, potassium, sodium, tromethamine and zinc salts. For a review on suitable salts, see Handbook of Pharmaceutical Salts: Properties, Selection, and Use by Stahl and Wermuth (Wiley-VCH, 2002).

Also included within the scope of the present invention are all stereoisomers, geometric isomers, and tautomeric forms of the disclosed compounds, including compounds exhibiting more than one type of isomerism, and mixtures of one or more thereof.

The present invention also includes all pharmaceutically acceptable isotopically-labelled analogs of the disclosed compounds, in which one or more atoms are replaced by atoms having the same atomic number, but an atomic mass or mass number different from the atomic mass or mass number which predominates in nature.

### E. METHODS OF TREATING CANCER HAVING A TUMOR WITH A MUTANT BAF CHROMATIN REMODELING COMPLEX

In one aspect, disclosed are methods of treating cancer in a subject in need thereof, the method comprising administering to the subject an effective amount of a disclosed compound, wherein the cancer comprises a tumor having a mutant BAF chromatin remodeling complex, thereby treating the condition. Examples of cancers for which the disclosed compounds, compositions, and methods can be useful include, but are not limited to, a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma).

Ewing sarcoma is the second most common primary bone tumor in children and accounts for approximately 2% of all childhood cancer diagnoses. The annual incidence of Ewing sarcoma is 2.93 children per 1,000,000. Approximately 200-250 children and adolescents in the United States are diagnosed with a tumor in the Ewing family of tumors each year. Two-thirds will be long-term survivors (more than five years). Ewing sarcoma affects males more often than females. It may affect individuals of any age, but most often occurs in individuals between 10 and 20 years of age.

Rhabdomyosarcoma (RMS) is a type of sarcoma that affects soft tissue such as muscles. RMS is rare, with about 500 people being diagnosed in the United States each year (Rhabdomyosarcoma: Causes, Symptoms, Treatment. Cleveland Clinic https://my.clevelandclinic.org/health/diseases/6226-rhabdomyosarcoma). Most of them are children or adolescents (RMS is the common type of soft tissue sarcoma in children), although it does sometimes occur in adults. Alveolar RMS is an aggressive type of RMS, and it occurs most frequently in the arms, legs, or torso.

According to GlobalData database, in 2019 the US accounted for 22.82% of the diagnosed incident cases of NSCLC in the 8 MM, with 181,422 cases. In the 8 MM, the number of diagnosed incident cases of NSCLC will grow at an Annual Growth Rate (AGR) of 3.01%, from 794,999 cases in 2019 to 1,034,554 cases by 2029 (30.13% growth over the forecast period).

Thus, in one aspect, disclosed are methods of treating cancer in a subject in need thereof, the method comprising administering to the subject an effective amount of a compound having a structure represented by a formula:

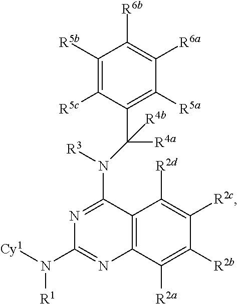

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{6a} and R^{6b} is independently selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof, wherein the cancer comprises a tumor having a mutant BAF chromatin remodeling complex.

Also disclosed are methods of treating cancer in a subject in need thereof, the method comprising administering to the subject an effective amount of a compound having a structure represented by a formula:

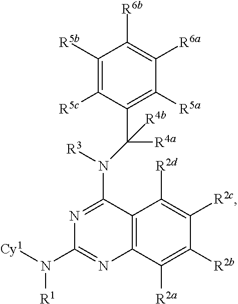

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R^{6a} is halogen; wherein R^{6b} is selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that either: (a) R^{6b} is chloro; (b) R^{6b} is hydrogen; or (c) R^{6b} is halogen and at least one of R^{5a}, R^{5b}, and R^{5c} is not hydrogen, or a pharmaceutically acceptable salt thereof, wherein the cancer comprises a tumor having a mutant BAF chromatin remodeling complex.

In various aspects, the compound has a structure represented by a formula:

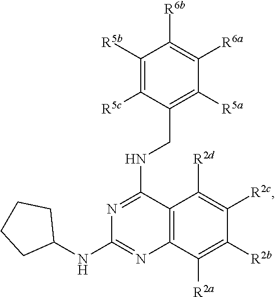

or a pharmaceutically acceptable salt thereof.

In various aspects, the compound is a structure selected from:

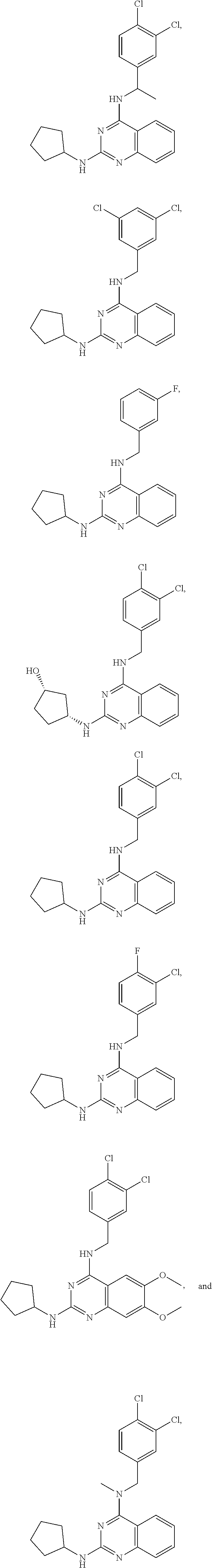

or a pharmaceutically acceptable salt thereof.

In various aspects, the compound is:

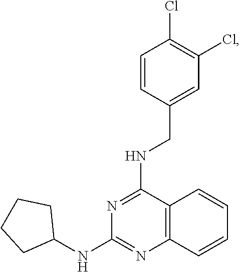 [SMILES: Clc1ccc(CNc2nc(NC3CCCC3)nc3ccccc23)cc1Cl]

or a pharmaceutically acceptable salt thereof.

In various aspects, R^{6a} is halogen. In a further aspect, R^{6b} is halogen. In a still further aspect, each of R^{6a} and R^{6b} is halogen.

In a further aspect, the subject has been diagnosed with a need for treatment of cancer prior to the administering step. In a still further aspect, the subject is at risk for developing cancer prior to the administering step.

In a further aspect, the subject is a mammal. In a still further aspect, the mammal is a human.

In a further aspect, the method further comprises the step of identifying a subject in need of treatment of cancer.

In various aspects, the cancer is selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma). In a further aspect, the cancer is selected from leukemia, neuroblastoma, melanoma, and triple-negative breast cancer.

In various aspects, the cancer is a pediatric cancer. In a further aspect, the pediatric cancer is leukemia, neuroblastoma, or melanoma.

In various aspects, the cancer is lung cancer. In a further aspect, the lung cancer is non-small cell lung adenocarcinoma.

In a further aspect, the effective amount is a therapeutically effective amount. In a still further aspect, the effective amount is a prophylactically effective amount.

In a further aspect, the method further comprises administering a chemotherapeutic agent to the subject. Examples of chemotherapeutic agents include, but are not limited to, alkylating agents (e.g., carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, streptozocin), antimetabolite agents (e.g., gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, thioguanine), antineoplastic antibiotic agents (e.g., doxorubicin, mitoxantrone, bleomycin, daunorubicin, dactinomycin, epirubicin, idarubicin, plicamycin, mitomycin, pentostatin, valrubicin), mitotic inhibitor agents (e.g., irinotecan, topotecan, rubitecan, cabazitaxel, docetaxel, paclitaxel, etopside, vincristine, ixabepilone, vinorelbine, vinblastine, teniposide), and mTor inhibitor agents (e.g., everolimus, siroliumus, temsirolimus).

In a further aspect, the compound and the agent are administered sequentially. In a still further aspect, the compound and the agent are administered simultaneously.

In a further aspect, the compound and the agent are co-formulated. In a still further aspect, the compound and the agent are co-packaged.

In a further aspect, the compound is administered as a single active agent.

### F. METHODS OF MODULATING EXPRESSION OF ONE OR MORE GENES SELECTED FROM SMARCA2 AND SMARCA4 IN A SUBJECT

In one aspect, disclosed are methods of modulating expression of one or more genes selected from SMARCA2 and SMARCA4 in a subject in need thereof, the method comprising administering to the subject an effective amount of a disclosed compound, thereby modulating the expression of one or more genes selected from SMARCA2 and SMARCA4 in the subject.

Thus, in one aspect, disclosed are methods of modulating expression of one or more genes selected from SMARCA2 and SMARCA4 in a subject, the method comprising administering to the subject an effective amount of a compound having a structure represented by a formula:

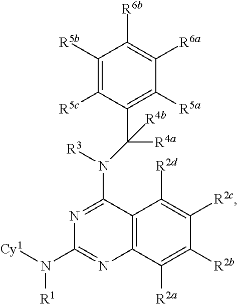

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R^{6a} is halogen; wherein R^{6b} is selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that either: (a) R^{6b} is chloro; (b) R^{6b} is hydrogen; or (c) R^{6b} is halogen and at least one of R^{5a}, R^{5b}, and R^{5c} is not hydrogen, or a pharmaceutically acceptable salt thereof.

In various aspects, the compound is selected from:

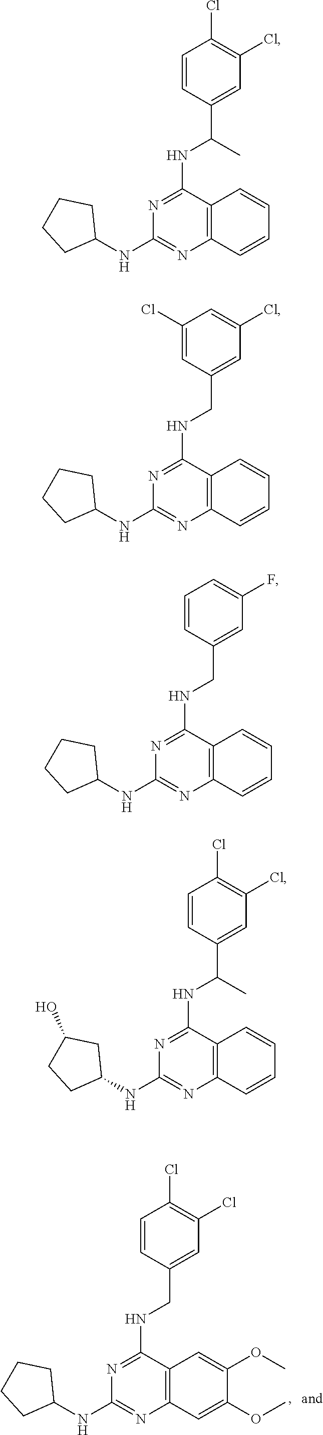 [SMILES: CC(Nc1nc(NC2CCCC2)nc2ccccc12)c1ccc(Cl)c(Cl)c1 ; Clc1cc(Cl)cc(CNc2nc(NC3CCCC3)nc3ccccc23)c1 ; Fc1cccc(CNc2nc(NC3CCCC3)nc3ccccc23)c1 ; CC(Nc1nc(N[C@@H]2CC[C@H](O)C2)nc2ccccc12)c1ccc(Cl)c(Cl)c1 ; COc1cc2nc(NC3CCCC3)nc(NCc3ccc(Cl)c(Cl)c3)c2cc1OC]

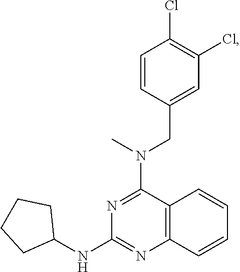 [SMILES: CN(Cc1ccc(Cl)c(Cl)c1)c1nc(NC2CCCC2)nc2ccccc12]

or a pharmaceutically acceptable salt thereof.

In various aspects, the compound is:

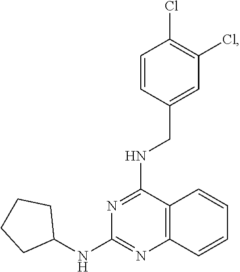 [SMILES: Clc1ccc(CNc2nc(NC3CCCC3)nc3ccccc23)cc1Cl]

or a pharmaceutically acceptable salt thereof.

In a further aspect, modulating is decreasing. In a still further aspect, modulating is inhibiting.

In a further aspect, the method modifies expression of SMARCA2. In a still further aspect, the method modifies expression of SMARC4. In yet a further aspect, the method modifies expression of SMARCA2 and SMARC4.

In a further aspect, the subject has been diagnosed with a disorder associated with altered expression of SMARCA2 and/or SMARCA4. In a still further aspect, the disorder is cancer.

In a further aspect, the subject has been diagnosed with the disorder prior to the administering step. In still a further aspect, the subject has been diagnosed with a need for modulating expression of one or more genes selected from of SMARCA2 and SMARCA4 prior to the administering step.

In a further aspect, the subject has been diagnosed with a need for treatment of cancer prior to the administering step. In an even further aspect, the cancer comprises a tumor having a mutant BAF chromatin remodeling complex.

In a further aspect, the method further comprises the step of identifying a subject in need of treatment of a disorder associated with altered expression of SMARCA2 and/or SMARCA4. In a still further aspect, the method further comprises the step of identifying a subject in need of treatment of cancer.

In a further aspect, the disorder associated with altered expression of SMARCA2 and/or SMARCA4 is cancer. Examples of cancers include, but are not limited to, sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, gliomas, leukemia, lymphoma, chronic myeloproliferative disorders, myelodysplastic syndrome, myeloproliferative neoplasm, non-small cell lung carcinoma, and plasma cell neoplasm (myeloma).

In a further aspect, the effective amount is a therapeutically effective amount. In a still further aspect, the effective amount is a prophylactically effective amount.

In a further aspect, the method further comprises the step of administering a therapeutically effective amount of at least one agent associated with the treatment of a disorder associated with altered expression of SMARCA2 and/or SMARCA4. In a still further aspect, the agent is a chemotherapeutic agent.

In a further aspect, the compound and the agent are administered sequentially. In a still further aspect, the compound and the agent are administered simultaneously.

In a further aspect, the compound and the agent are co-formulated. In a still further aspect, the compound and the agent are co-packaged.

In a further aspect, the compound is administered as a single active agent.

### G. METHODS OF MODULATING EXPRESSION OF ONE OR MORE GENES SELECTED FROM SMARCA2 AND SMARCA4 IN A CELL

In one aspect, disclosed are methods of modulating expression of one or more genes selected from SMARCA2 and/or SMARC4 in a cell, the method comprising contacting the cell with an effective amount of a disclosed compound, thereby modulating expression of SMARCA2 and/or SMARCA4 signaling in the cell.

Thus, in one aspect, disclosed are methods of modulating expression of one or more genes selected from SMARCA2 and SMARC4 in a cell, the method comprising contacting the cell with an effective amount of a compound having a structure represented by a formula:

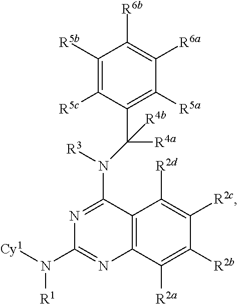

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R^{6a} is halogen; wherein R^{6b} is selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that either: (a) R^{6b} is chloro; (b) R^{6b} is hydrogen; or (c) R^{6b} is halogen and at least one of R^{5a}, R^{5b}, and R^{5c} is not hydrogen, or a pharmaceutically acceptable salt thereof.

In various aspects, the compound is a structure selected from:

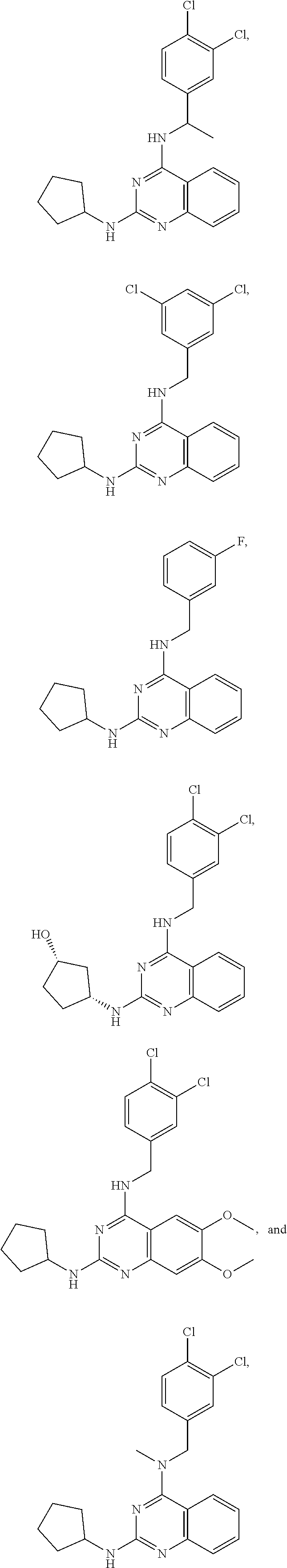 [SMILES: CC(Nc1nc(NC2CCCC2)nc2ccccc12)c1ccc(Cl)c(Cl)c1 ; Clc1cc(Cl)cc(CNc2nc(NC3CCCC3)nc3ccccc23)c1 ; Fc1cccc(CNc2nc(NC3CCCC3)nc3ccccc23)c1 ; O[C@H]1CC[C@@H](Nc2nc(NCc3ccc(Cl)c(Cl)c3)c3ccccc3n2)C1 ; COc1cc2nc(NC3CCCC3)nc(NCc3ccc(Cl)c(Cl)c3)c2cc1OC ; CN(Cc1ccc(Cl)c(Cl)c1)c1nc(NC2CCCC2)nc2ccccc12]

or a pharmaceutically acceptable salt thereof.

In various aspects, the compound is:

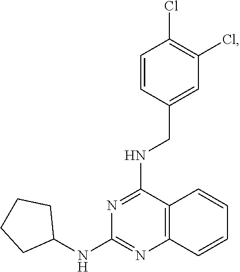 [SMILES: Clc1ccc(CNc2nc(NC3CCCC3)nc3ccccc23)cc1Cl]

or a pharmaceutically acceptable salt thereof.

In a further aspect, modulating is decreasing. In a still further aspect, modulating is inhibiting.

In a further aspect, the method modulates expression of SMARC2. In a still further aspect, the method modulates expression of SMARC4. In yet a further aspect, the method modulates expression of SMARC2 and SMARCA4.

In a further aspect, the cell is mammalian. In a still further aspect, the cell is human.

In a further aspect, the cell has been isolated from a human prior to the administering step.

In a further aspect, contacting is via administration to a subject. In a still further aspect, the subject has been diagnosed with a need for modulation of the expression of one or more genes selected from SMARCA2 and SMARCA4 prior to the administering step. In yet a further aspect, the subject has been diagnosed with a need for treatment of a disorder associated with altered expression of SMARCA2 and/or SMARCA4.

In a further aspect, the subject has been diagnosed with a need for treatment of cancer prior to the administering step.

In a further aspect, the disorder associated with altered expression of SMARCA2 and/or SMARCA4 is cancer. Examples of cancers include, but are not limited to, sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, gliomas, leukemia, lymphoma, chronic myeloproliferative disorders, myelodysplastic syndrome, myeloproliferative neoplasm, non-small cell lung carcinoma, and plasma cell neoplasm (myeloma).

### H. ADDITIONAL METHODS OF USING THE COMPOUNDS

The compounds and pharmaceutical compositions of the invention are useful in treating or controlling disorders associated with altered expression of SMARCA2 and/or SMARCA4 such as, for example, cancer (e.g., sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, gliomas, leukemia, lymphoma, chronic myeloproliferative disorders, myelodysplastic syndrome, myeloproliferative neoplasm, non-small cell lung carcinoma, plasma cell neoplasm (myeloma)).

To treat or control the condition, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a disorder associated with altered expression of SMARCA2 and/or SMARCA4 such as, for example, cancer (e.g., sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, gliomas, leukemia, lymphoma, chronic myeloproliferative disorders, myelodysplastic syndrome, myeloproliferative neoplasm, non-small cell lung carcinoma, plasma cell neoplasm (myeloma)).

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a disorder associated with altered expression of SMARCA2 and/or SMARCA4 such as, for example, cancer (e.g., sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, gliomas, leukemia, lymphoma, chronic myeloproliferative disorders, myelodysplastic syndrome, myeloproliferative neoplasm, non-small cell lung carcinoma, plasma cell neoplasm (myeloma)).

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

### 1. Use of Compounds

In one aspect, the invention relates to the use of a disclosed compound or a product of a disclosed method. In a further aspect, a use relates to the manufacture of a medicament for the treatment of a disorder associated with altered expression of SMARCA2 and/or SMARCA4 such as, for example, cancer (e.g., sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, plasma cell neoplasm (myeloma)).

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a disorder associated with altered expression of SMARCA2 and/or SMARCA4 in a subject. In one aspect, the use is characterized in that the subject is a human. In one aspect, the use is characterized in that the disorder associated with altered expression of SMARCA2 and/or SMARCA4 is cancer.

In various aspects, the use relates to a treatment of cancer comprising a tumor having a mutant BAF chromatin remodeling complex.

In a further aspect, the use relates to the manufacture of a medicament for the treatment of a disorder associated with altered expression of SMARCA2 and/or SMARCA4 in a subject.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a disorder associated with altered expression of SMARCA2 and/or SMARCA4 in a mammal. In a further aspect, the disorder associated with altered expression of SMARCA2 and/or SMARCA4 is a cancer (e.g., sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, plasma cell neoplasm (myeloma)). In a further aspect, the cancer comprises a tumor having a mutant BAF chromatin remodeling complex.

### 2. Manufacture of a Medicament

In one aspect, the invention relates to a method for the manufacture of a medicament for treating a disorder associated with altered expression of SMARCA2 and/or SMARCA4 in a subject having the condition, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the treatment of a disorder associated with altered expression of SMARCA2 and/or SMARCA4 (e.g., cancer such as, for example, sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, plasma cell neoplasm (myeloma)). The dose administered to an animal, particularly a human, in the context of the present invention should be sufficient to affect a therapeutic response in the animal over a reasonable timeframe. One skilled in the art will recognize that dosage will depend upon a variety of factors including the condition of the animal and the body weight of the animal.

The total amount of the compound of the present disclosure administered in a typical treatment is preferably between about 0.05 mg/kg and about 100 mg/kg of body weight for mice, and more preferably between 0.05 mg/kg and about 50 mg/kg of body weight for mice, and between about 100 mg/kg and about 500 mg/kg of body weight for humans, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing, and frequency of administration as well as the existence, nature and extent of any adverse side effects that might accompany the administration of the compound and the desired physiological effect. It will be appreciated by one of skill in the art that various conditions or disease states, in particular chronic conditions, or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### 3. Kits

In one aspect, disclosed are kits comprising a disclosed compound, and one or more of: (a) a chemotherapeutic agent; (b) instructions for administering the compound in connection with treating cancer; and (c) instructions for treating cancer.

Thus, in one aspect, disclosed are kits comprising a compound having a structure represented by a formula:

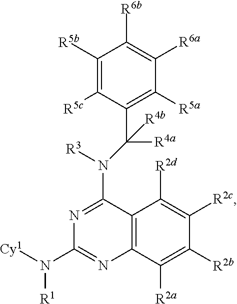

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{6a} and R^{6b} is independently selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof, and one or more selected from: (a) a chemotherapeutic agent; (b) instructions for administering the compound in connection with treating cancer; and (c) instructions for treating cancer.

In a further aspect, disclosed are kits comprising a compound having a structure represented by a formula:

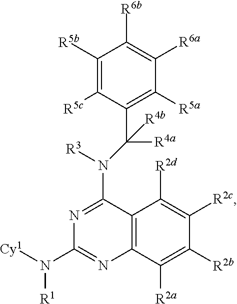

wherein R¹ is selected from hydrogen and C1-C4 alkyl; wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R³ is selected from hydrogen and C1-C4 alkyl; wherein each of R^{4a} and R^{4b} is independently selected from hydrogen, halogen, and C1-C4 alkyl; wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein R^{6a} is halogen; wherein R^{6b} is selected from hydrogen and halogen; and wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that either: (a) R^{6b} is chloro; (b) R^{6b} is hydrogen; or (c) R^{6b} is halogen and at least one of R^{5a}, R^{5b}, and R^{5c} is not hydrogen, or a pharmaceutically acceptable salt thereof, and one or more selected from: (a) a chemotherapeutic agent; (b) instructions for administering the compound in connection with treating cancer; and (c) instructions for treating cancer.

In various aspects, the compound is selected from:

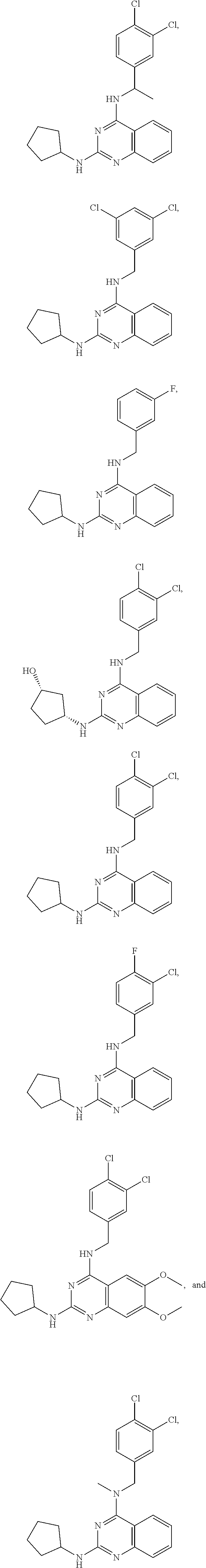

or a pharmaceutically acceptable salt thereof.

In various aspects, the compound is:

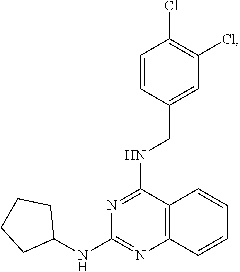 [SMILES: Clc1ccc(CNc2nc(NC3CCCC3)nc3ccccc23)cc1Cl]

or a pharmaceutically acceptable salt thereof.

In a further aspect, the kit comprises the chemotherapeutic agent. Examples of chemotherapeutic agents include, but are not limited to, alkylating agents, antimetabolite agents, antineoplastic antibiotic agents, mitotic inhibitor agents, and mTor inhibitor agents.

In various aspects, the chemotherapeutic agent is an alkylating agent. In a further aspect, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In various aspects, the chemotherapeutic agent is an antimetabolite agent. In a further aspect, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In various aspects, the chemotherapeutic agent is an antineoplastic antibiotic agent. In a further aspect, the antineoplastic antibiotic agent is selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In various aspects, the chemotherapeutic agent is a mitotic agent. In a further aspect, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In various aspects, the chemotherapeutic agent is a mTor inhibitor agent. In a further aspect, the mTor inhibitor agent is selected from everolimus, siroliumus, and temsirolimus, or a pharmaceutically acceptable salt thereof.

In a further aspect, the compound and the agent are co-formulated. In a further aspect, the compound and the agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### I. EXAMPLES

To fit inside the nucleus of eukaryotic cells, DNA is packaged with the help of histone proteins in a specific 3D structure called chromatin. To properly transcribe, replicate, and repair DNA, a cell needs to actively remodel chromatin. One of the main ways used by the cell to do so is the use of ATP-dependent chromatin remodeling complexes. Like the name suggests, these complexes utilize the energy derived from ATP hydrolysis to modify the chromatin structure, promoting or restricting access to specific regions of the DNA by other molecules.

The first chromatin remodeling complex identified was the BAF complex (BRG1/BRM Associated Factors, also known as SWI/SNF). BAF represents a family of complexes made up of 11-15 different proteins in mammals (Clapier and Cairns (2009) Annu Rev Biochem 78: 273-304; Hargreaves and Crabtree (2011) Cell Res 21: 396-420). The enzymatic motor of the complexes are two mutually exclusive helicases/ATPases of the SNF2 family called SMARCA2 (also called BRAHMA, BRM) and SMARCA4 (also called BRAHMA Related Gene 1, BRG1) (Guerrero-Martinez and Reyes (2018) Sci Rep 8: 2043). SMARCA2/4 serve an essential role in the BAF complexes by generating the energy required for chromatin remodeling through ATP hydrolysis.

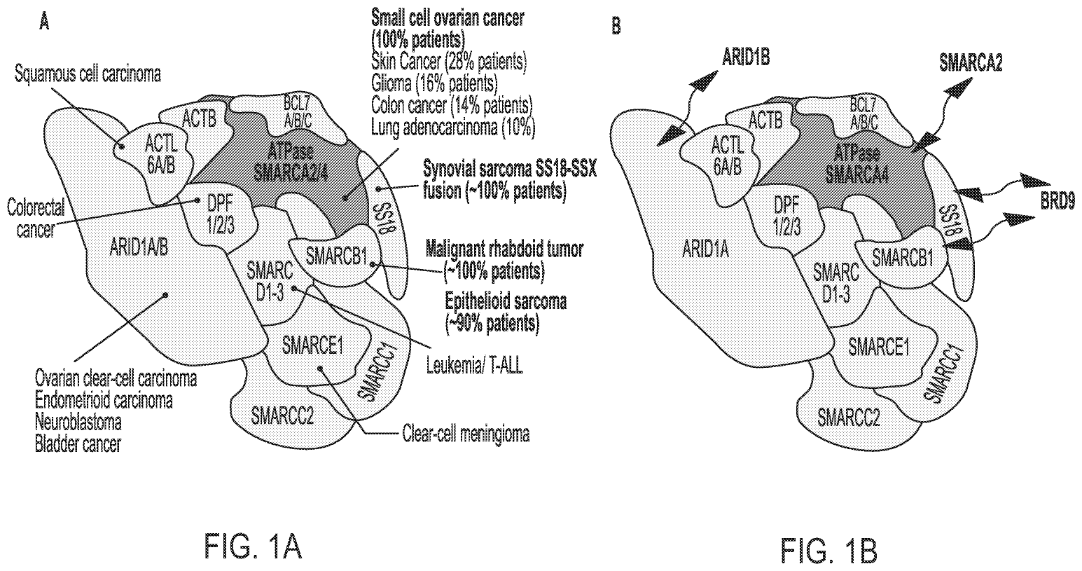

Outside of their central role in healthy cells, BAF complexes are very relevant to cancer development, and genetic screens confirmed the presence of mutations in BAF subunits in about 20% of all human cancers (Kadoch et al. (2013) Nat Genet 45: 592-601). Moreover, recent studies have also clarified the oncogenic role of BAF complexes and molecular relationship of an EWSFLI1 and BAFs in Ewing's Sarcoma (EwS) (Boulay et al. (2017) Cell 171: 163-178.e19; Jayabal, P. et al. (2021) Cell Reports 36: 109254). SMARCA4 is the ATPase subunit more frequently mutated and it is silenced in several cancer types, leading to its initial consideration as a tumor suppressor gene. However, recent studies have linked the overexpression of SMARCA4 to oncogenesis and tumor maintenance in several tumor types (Guerrero-Martinez and Reyes (2018) Sci Rep 8: 2043) (e.g., leukemia (Shi, J. et al. (2013) Genes Dev 27: 2648-2662; Buscarlet, M. et al. (2014) Blood 123: 1720-1728), TNBC (Wu et al. (2015) J. Cell Physiol 230: 2683-2694; Wu et al. (2016) Oncotarget 7: 27158-27175), melanoma (Keenen et al. (2010) Oncogene 29: 81-92), and neuroblastoma (Jubierre et al. (2016) Oncogene 35: 5179-5190)) (FIG. 1A-B). While SMARCA2 is not as frequently mutated in tumors as SMARCA4, it was shown to be silenced in a number of cancer cell lines (Guerrero-Martinez and Reyes (2018) Sci Rep 8: 2043).

Because these SMARCA2/4 alterations are typically loss-of-function mutations, they are not directly therapeutically targetable. As a result, major efforts have been directed towards the identification of other vulnerabilities created by these mutations (Helming et al. (2014) Cancer Cell 26: 309-317). A major breakthrough was the discovery that SMARCA4 deficient cancer cells are highly sensitive to SMARCA2 depletion (synthetic lethality), probably as a result of residual BAF complex in SMARCA4-mutated cells relying on the activity of SMARCA2 (Hoffman et al. (2014) PNAS 111: 3128-3133; Oike et al. (2013) Cancer Res 73: 5508-5518; Wilson et al. (2014) Molecular and Cellular Biology 34: 1136-1144). The dependence of SMARCA4 mutant cancer cells on SMARCA2 activity makes inhibition of the latter an ideal target for cancer treatment (Helming et al. (2014) Cancer Cell 26: 309-317).

Only a fraction of cancer types possesses activated oncogenes, whereas most cancers have genetic changes associated with loss-of-function mutations (LOFs). The inactivated gene products generated from LOFs are often not adapt for targeting by small molecule therapeutics. However, LOFs can sometimes cause new vulnerabilities in the form of synthetic lethality. One example of this process is the lethality of SMARCA2 inhibition in many cancers with mutant SMARCA4. As a result, compounds that inhibit SMARCA2 have significant therapeutic potential in cancers with SMARCA4 LOFs.

As detailed herein, AT-301 is a synthetic molecule that inhibits SMARCA2 by targeting the ATPase pocket, and it is active in vitro and in vivo. AT-301 was already shown to attenuate the growth of nine Ewing's sarcoma cells lines, and four alveolar Rhabdomyosarcoma tested so far. AT-301 presents the following additional advantages: (1) a study identified the SMARCA2 ATPase domain as the most relevant target (as opposed to the bromodomain) to abolish cell proliferation in SMARCA4 mutant cancer cells (Vangamudi et al. (2015) Cancer Res 75: 3865-3878); (2) AT-301 shows on-target inhibition as it fails to inhibit the activity of other SWI/SNF ATPases that are not part of the BAF complex in human cells, such as SMARCAL1; and (3) SMARCA2 does not seem to play a significant role in normal functioning of a cell as evident from fully viable SMARCA2 KO mice (although some weight gain and alteration in cell cycle control of mouse-derived embryonic fibroblasts was reported) (Reyes et al. (1998) EMBO J 17: 6979-6991). Therefore, targeting SMARCA2 by small molecules should have minimal impact on normal cellular function.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Chemistry Experimentals

All reactions were carried out in an oven-dried glassware under argon atmosphere using standard gas-tight syringe, cannula, and septa. The reaction temperatures were measured externally. Stirring was achieved with oven dried magnetic bars. All the reactions were done in anhydrous solvents (CH₂Cl₂, THF, MeOH) purchased from Sigma-Aldrich. All commercially purchased reagents were used without purification. The reactions were monitored by thin-layer chromatography (TLC) on a pre-coated silica gel (60 F254) glass plates from EMD Millipore and visualized using UV light (254 nm). Purification of the compounds was performed on Teledyne-ISCO Combiflash Rf 200 purification system using Redisep Rf® normal phase silica gel columns 230-400 mesh. ESI-MS spectra were recorded on a BioTof-2 time-of-flight mass spectrometer. Proton NMR spectra were recorded on a Varian Unity 400 NMR spectrometer operating at 400 MHz calibrated to the solvent peak and TMS peak. The chemical formula and Exact Mass for target compounds were determined from the (M+H)⁺ by high resolution mass spectroscopy using an Agilent 6210 Electrospray Time of Flight.

### a. General Experimental

All operations were carried out at room or ambient temperature, that is, in the range of 18-25° C.; evaporation of solvent was carried out using a rotary evaporator under reduced pressure with a bath of up to 50° C.; reactions were monitored by thin layer chromatography (tlc) and reaction times are given for illustration only. Unless otherwise indicated all reactions were conducted in standard commercially available glassware using standard synthetic chemistry methods and setup. All air- and moisture-sensitive reactions were performed under nitrogen atmosphere with dried solvents and glassware under anhydrous conditions. Starting materials and reagents were commercial compounds of the highest purity available and were used without purification (See list of specific reagents below). Solvents used for reactions were indicated as of commercial dry or extra-dry or analytical grade. Analytical thin layer chromatography was performed on aluminum plates coated with Merck Kieselgel 60F254 and visualized by UV irradiation (254 nm) or by staining with a solution of potassium permanganate. Flash column chromatography was performed on Biotage Isolera One 2.2 using commercial columns that were pre-packed with Merck Kieselgel 60 (230-400 mesh) silica gel. Final compounds for biological testing are all ≥95% purity as determined by HPLC-MS and ¹H NMR. ¹H NMR experiments were recorded on Agilent DD2 400 MHz spectrometers at ambient temperature. Samples were dissolved and prepared in deuterated solvents (CDCl₃, CD₃OD and DMSOd₆) with residual solvents being used as the internal standard in all cases. All deuterated solvent peaks were corrected to the standard chemical shifts (CDCl₃, dH=7.26 ppm; CD₃OD, dH=3.31 ppm; DMSOd₆, dH=2.50 ppm). Spectra were all manually integrated after automatic baseline correction. Chemical shifts (d) are given in parts per million (ppm), and coupling constants (J) are given in Hertz (Hz). The proton spectra are reported as follows: d (multiplicity, coupling constant J, number of protons). The following abbreviations were used to explain the multiplicities: app=apparent, b=broad, d=doublet, dd=doublet of doublets, ddd=doublet of doublet of doublets, dddd=doublet of doublet of doublet of doublets, m=multiplet, s=singlet, t=triplet. All samples were analyzed on Agilent 1290 series HPLC system comprised of binary pumps, degasser and UV detector, equipped with an auto-sampler that is coupled with Agilent 6150 mass spectrometer. Purity was determined via UV detection with a bandwidth of 170 nm in the range from 230-400 nm. The general LC parameters were as follows: Column—Zorbax Eclipse Plus C18, size 2.1×50 mm; Solvent A: 0.10% formic acid in water, Solvent B: 0.00% formic acid in acetonitrile; Flow rate—0.7 mL/min; Gradient: 5% B to 95% B in 5 min and hold at 95% B for 2 min; UV detector—channel 1=254 nm, channel 2=254 nm. Mass detector Agilent Jet Stream—Electron Ionization (AJS-ES).

The following abbreviations are used:
THF: tetrahydrofuran
DCM or CH₂Cl₂: dichloromethane
DCE: dichloroethane
NaHCO₃. sodium bicarbonate
HCl: hydrogen chloride
MgSO₄: magnesium sulfate
Na₂SO₄: sodium sulfate
DME: dimethoxyethane
n-BuLi: n-butyllithium
DMF: dimethylformamide
DMSO: dimethylsulfoxide
Et₂O: diethyl ether
MeOH: methanol
EtOAc: ethyl acetate

### b. Representative Synthesis of N²-cyclopentyl-N⁴-(3,4-dichlorobenzyl)quinazoline-2,4-diamine (AT-301; No. 38)

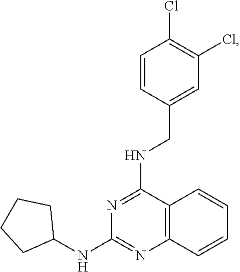 [SMILES: Clc1ccc(CNc2nc(NC3CCCC3)nc3ccccc23)cc1Cl]

2-chloro-N-(3,4-dichlorobenzyl)quinazolin-4-amine (3): To a stirring solution of 2,4-dichloroquinazoline (5.0 g, 25.1 mmol) in THF (125 mL) at room temperature was added 3,4-dichlorobenzylamine (4.0 mL, 30.3 mmol). The mixture was stirred for 24 hr at room temperature, during which time a precipitate formed. The slurry was filtered and washed with hexanes. The collected filtrate was slurried with DCM/hexanes and filtered. The filtrate was washed with hexanes, collected and dried under reduce vacuum to yield 4.4 g (52% yield) of 2-chloro-N-(3,4-dichlorobenzyl)quinazolin-4-amine (3) as a white powder.

N²-cyclopentyl-N⁴-(3,4-dichlorobenzyl)quinazoline-2,4-diamine (AT-301; 38): To a 30 mL microwave reaction vial was added 2-chloro-N-(3,4-dichlorobenzyl)quinazolin-4-amine 3 (1.0 g, 2.95 mmol), sec-butanol (15 mL) and cyclopentylamine (251 mg, 2.95 mmol). The mixture was irradiated in an Anton Par microwave reactor for 30 minutes at 180° C. The reaction was cooled to room temperature, during which time a precipitate formed. The solids were filtered, washed with hexanes and dried under reduced pressure to yield N²-cyclopentyl-N⁴-(3,4-dichlorobenzyl)quinazoline-2,4-diamine (970 mg, 85% yield) as a white powder.

### c. Additional Exemplary Compounds

Compounds listed in Table 1 below were prepared as detailed above for AT-301.

TABLE 1

| No. | Structure | MW |
|---|---|---|
| 1 | 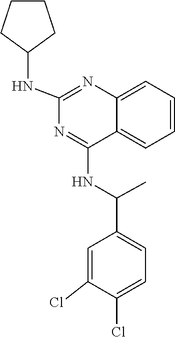 [SMILES: CC(Nc1nc(NC2CCCC2)nc2ccccc12)c1ccc(Cl)c(Cl)c1] | 401.3 |
| 2 | 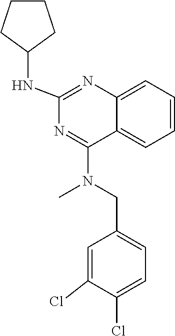 [SMILES: CN(Cc1ccc(Cl)c(Cl)c1)c1nc(NC2CCCC2)nc2ccccc12] | 401.3 |
| 3 | 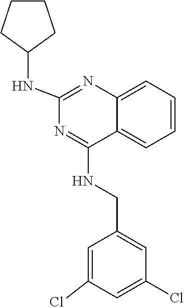 [SMILES: Clc1cc(Cl)cc(CNc2nc(NC3CCCC3)nc3ccccc23)c1] | 387.3 |
| 4 | 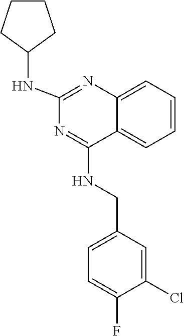 [SMILES: Fc1ccc(CNc2nc(NC3CCCC3)nc3ccccc23)cc1Cl] | 370.9 |
| 5 | 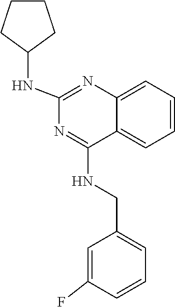 [SMILES: Fc1cccc(CNc2nc(NC3CCCC3)nc3ccccc23)c1] | 336.4 |
| 6 | 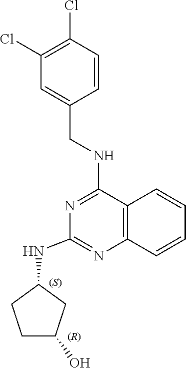 | 403.3 |
| 7 | 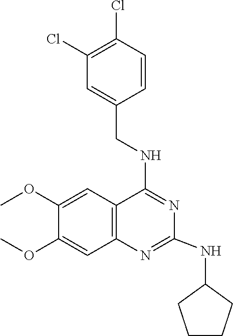 [SMILES: COc1cc2nc(NC3CCCC3)nc(NCc3ccc(Cl)c(Cl)c3)c2cc1OC] | 447.4 |
| 8 | 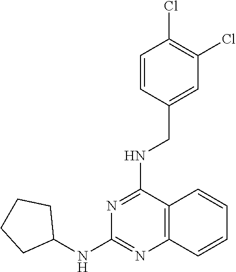 [SMILES: Clc1ccc(CNc2nc(NC3CCCC3)nc3ccccc23)cc1Cl] | 387.3 |
| 9 | 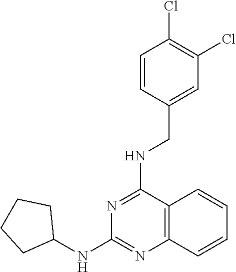 [SMILES: Clc1ccc(CNc2nc(NC3CCCC3)nc3ccccc23)cc1Cl] | 387.3 |

### 2. Purification of Human SMARCA2 Enzyme, DNA Substrate, Enzymatic Assay, and High-Throughput Screen

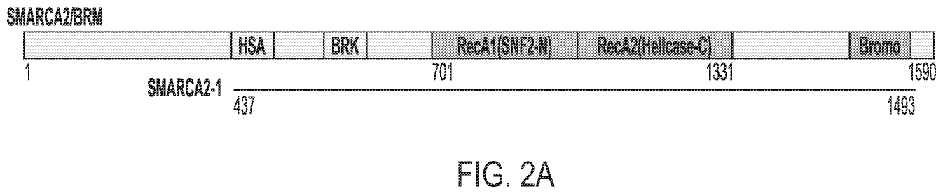

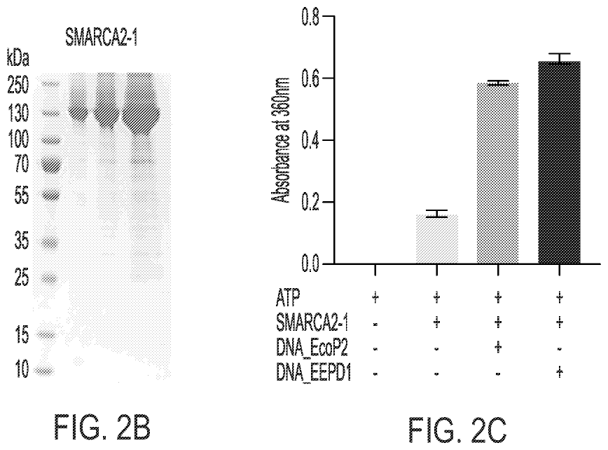

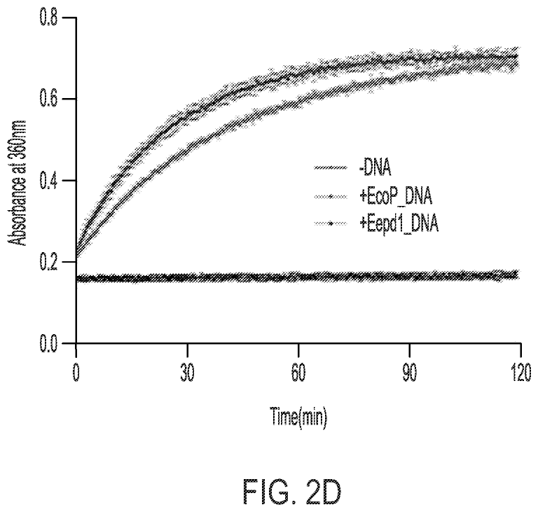

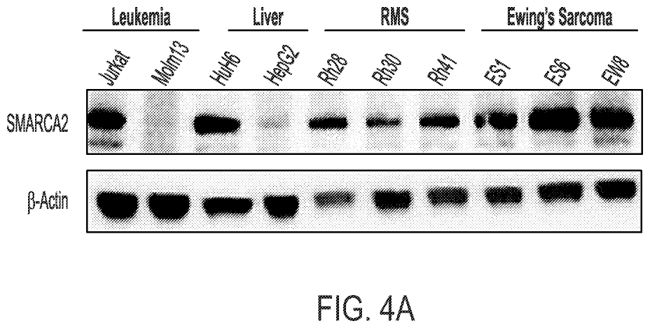

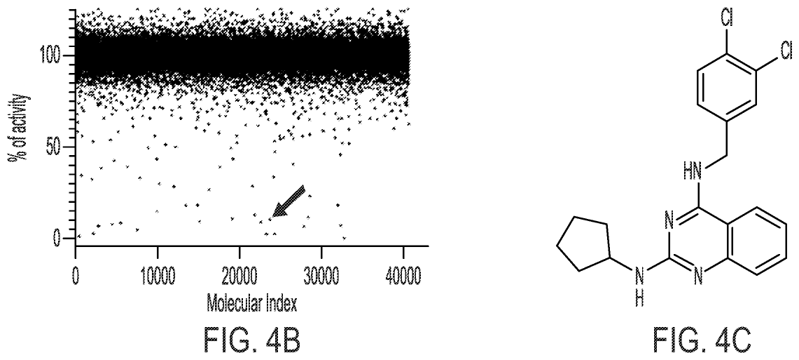

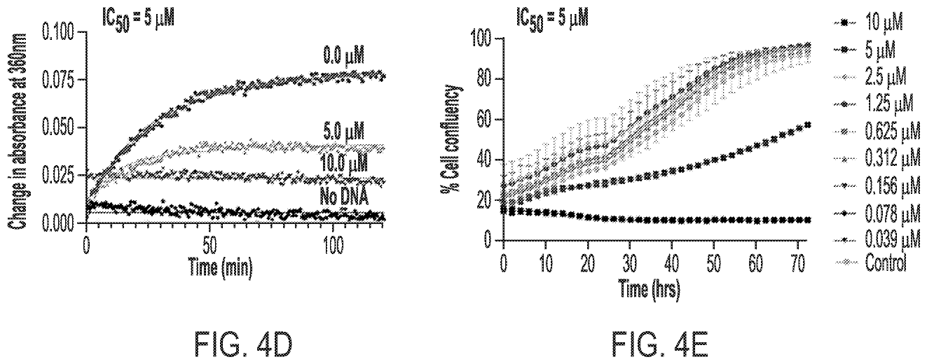

The SMARCA2-1 gene (amino acids 437-1493) encompassing all domains (HAS, BRK, ATPase and Bromodomain) required for DNA binding and ATPase activity were cloned into a pFASTBac1 plasmid for insect cell expression (FIG. 2A). In brief, one positive colony was selected and grown to produce enough bacmid DNA. The purified bacmid DNA was used to transfect expiSF9 insect cells using ExpiFectamine protocol as recommended by the manufacturer (Invitrogen). The P0 virus was first generated from the transfected cells, which was subsequently used to generate a P1 virus stock to have enough virus for several rounds of infections and/or large-scale production of SMARCA2-1 enzyme. Next, the experimental conditions were optimized for highest expression of this enzyme and the cultures scaled up to express and purify the enzyme by successive passage of soluble fraction through affinity, ion-exchange, and size exclusion chromatography steps (FIG. 2B). Next, a 29-mer double stranded DNA termed as DNA_EEPD1 (sequence below) was used to measure ATPase activity of SMARCA2 (FIG. 2C and FIG. 2D) and its inhibition by the small molecules described herein (FIG. 4D).

5′- CATGCAGCTGATGCGTACAACACACTGTG - 3′
3′- GTACGTCGACTACGCCCCCCCCCCCTTTT - 3′

This sequence does not resemble any known human DNA sequence but exhibited high signal in the ATPase assay (FIG. 2D). In brief, the disclosed ATPase assay is based on the inorganic phosphate (Pi) release from the enzymatic reaction, wherein the substrate 2-amino-6-mercapto-7-methylpurine ribose (MESG) is converted by purine phosphorylase (PNP) to ribose 1-phosphate and 2-amino-6-mercapto-7-methylpurine. This conversion changes the spectrophotometric shift of MSEG absorbance from 330 nm to 360 nm (FIG. 1C and FIG. 1D).

### 3. Evaluation of Inhibiting Activity Against SMARCA2

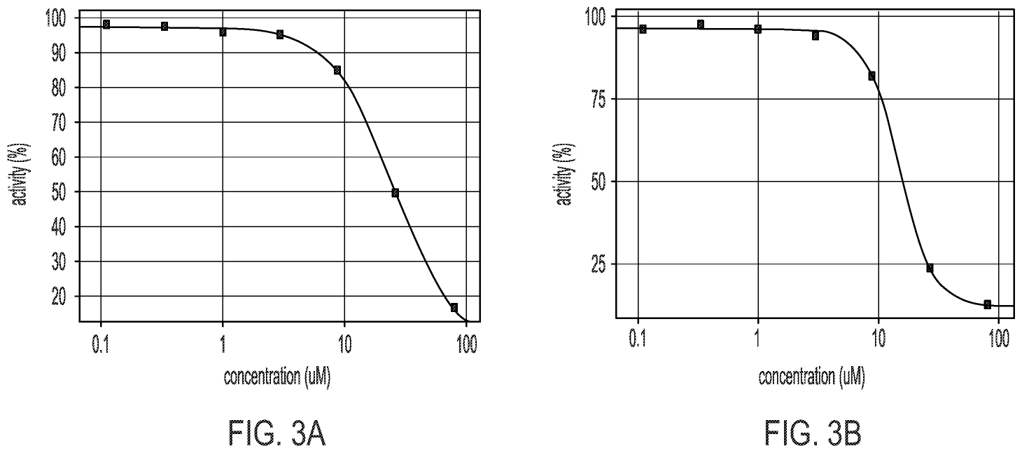

Exemplary compounds were evaluated for their ability to inhibit human SMARCA2 in ATPase assays as detailed in Table 2 below. Dose response curves for compound nos. 7 and 8 are shown in FIG. 3A and FIG. 3B.

TABLE 2

| | Dose response | ATPase inhibition | |
|---|---|---|---|
| | experiment | in a secondary | % ATPase |
| No. | (IC₅₀ in μM) | assay (Y/N) | activity in HTS |
| 1 | | Y | Not present in |
| | | | the HTS libraries |
| 2 | | Y | Not present in |
| | | | the HTS libraries |
| 3 | | Y | Not present in |
| | | | the HTS libraries |
| 4 | | | Not present in |
| | | | the HTS libraries |
| 5 | | | Not present in |
| | | | the HTS libraries |
| 6 | | | 97.3 |
| 7 | 25.3 | Y | 26.9 |
| 8 | 14.8 | Y (IC₅₀ = 5 μM) | 10.6 |
| 9 | | Y | Not present in |
| | | | the HTS libraries |

### 4. Evaluation of AT-301 Activity

Briefly, human SMARCA2 was purified, and a high-throughput screen (HTS) of 40,000 compounds was conducted. One of the top results, CIDD-0149689 (N2-cyclopentyl-N4-[(3,4-dichlorophenyl)methyl]quinazoline-2,4-diamine) or AT-301, identified from the HTS shows inhibitory potential against human SMARCA2 in two different ATPase assays (FIG. 4B-D). When tested on tumor cell lines, AT-301 strongly attenuated the growth of 8 different Ewing Sarcoma cell lines that showed increased SMARCA2 levels (FIG. 4A and FIG. 4E) with an IC₅₀ of ˜5 μM. Additionally, AT-301 also strongly attenuated the growth of 4 alveolar Rhabdomyosarcoma cell lines, and moderately attenuated the growth of 2 other lines. In brief, cells were grown in RPMI media supplemented with 10% Fetal bovine serum (FBS), 1× penicillin, and streptomycin antibiotics. Cells were placed in an incubator with 5% C02 and 50% humidity. When cells reached 90% confluency, cells were harvested, washed twice in 1×PBS, counted using trypan blue exclusion method. Then cells were seeded in 96-well plates (duplicates, n=2) at the density of 20,000 cells per well. Allowed them to attach to the bottom of the well for 1 to 2 hrs and treated with different concentration of AT-301 compound starting at highest concentration of 10 micromolar to serial dilution of the compound to the lowest concentration of 0.039 micromolar. Cells treated with only DMSO were also included as negative control. Cells were monitored for 72 hrs. by live cell imaging method (Incucyte S3) taking images every two hours. The confluency was determined and plotted against time (hours) by using Prism software.

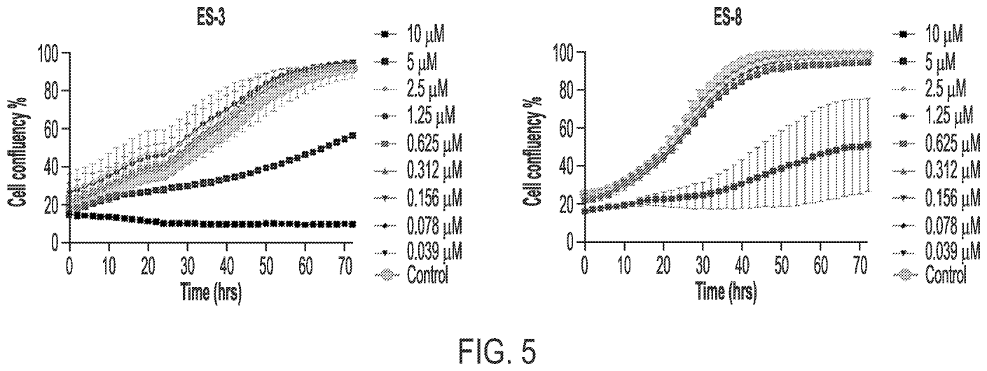

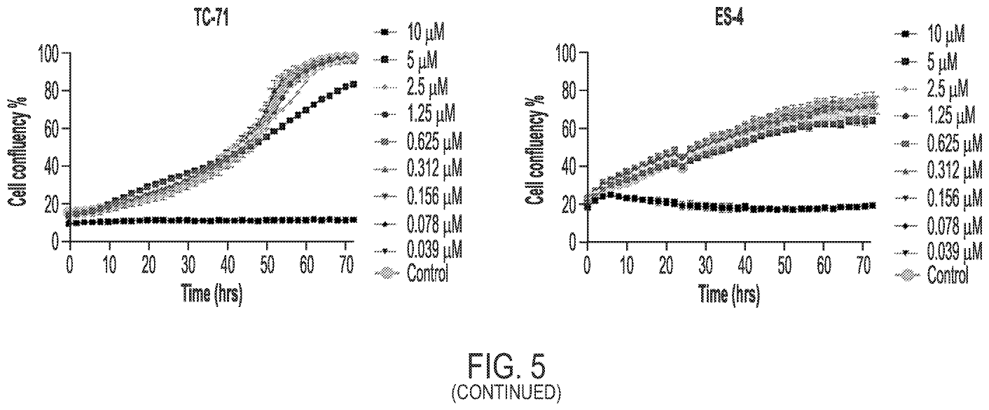

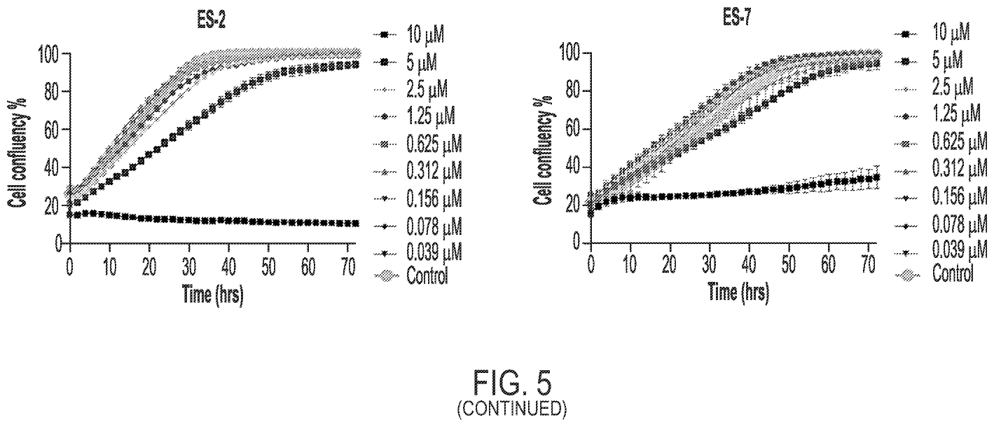

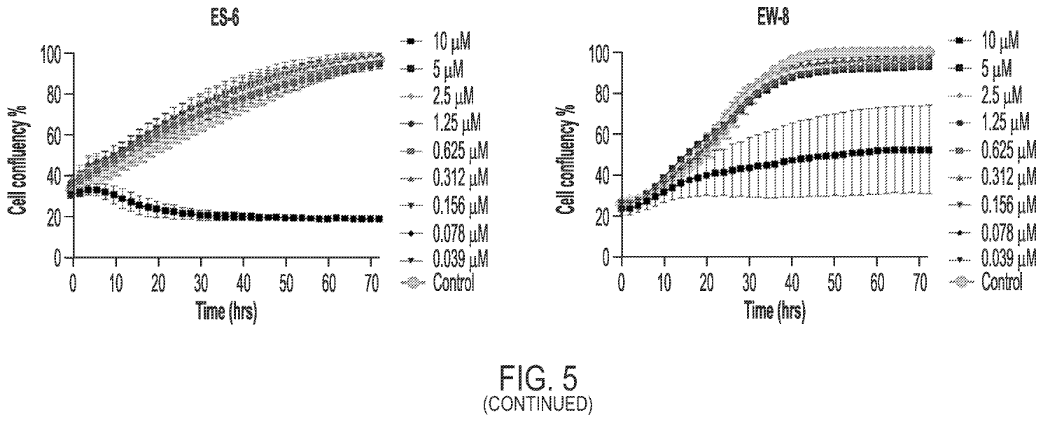

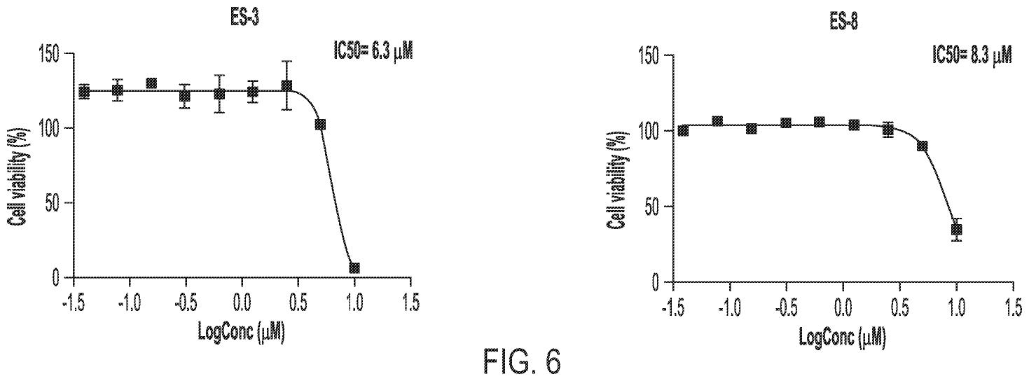

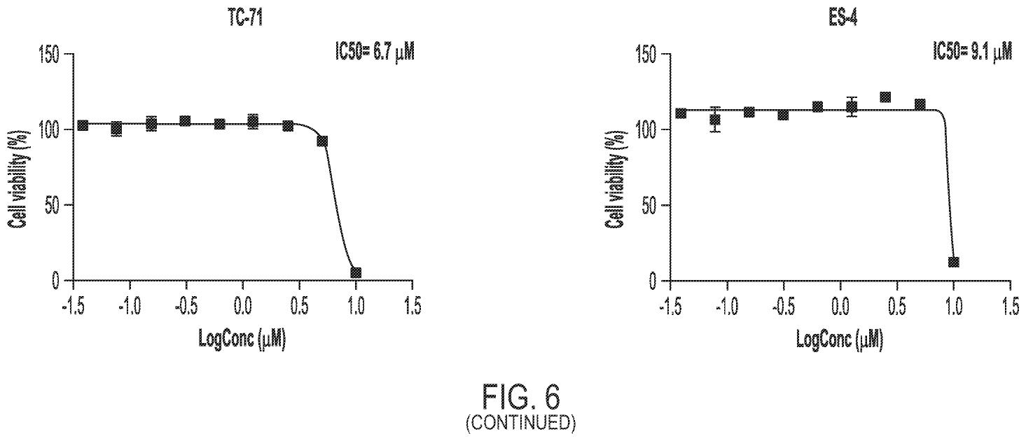

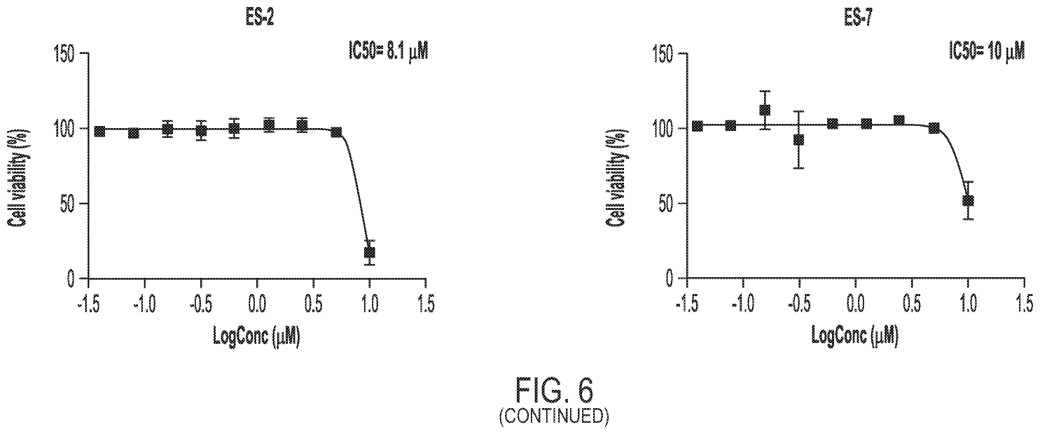

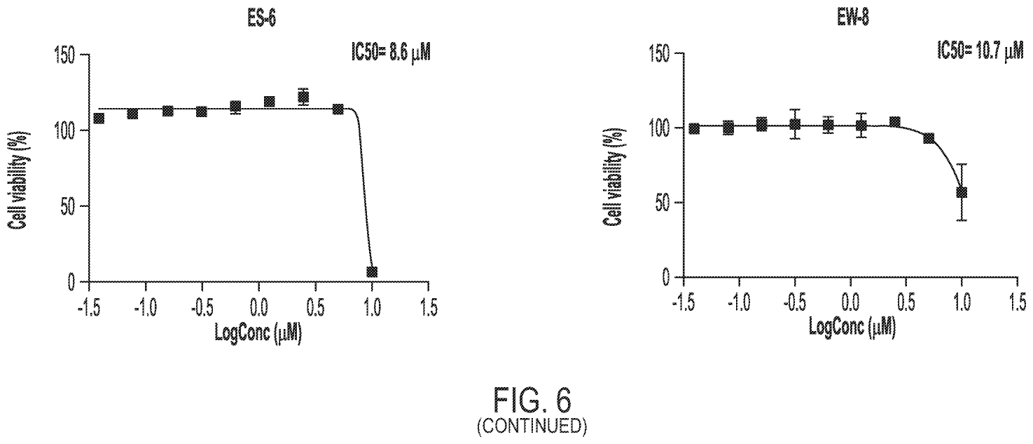

The change in % confluency of Ewing's sarcoma cells upon treatment with AT-301 is shown in FIG. 5. The change in % cell viability of Ewing's sarcoma cells post-treatment (72 hours) with AT-301 is shown in FIG. 6. Assays were performed as detailed above for FIG. 4A-E.

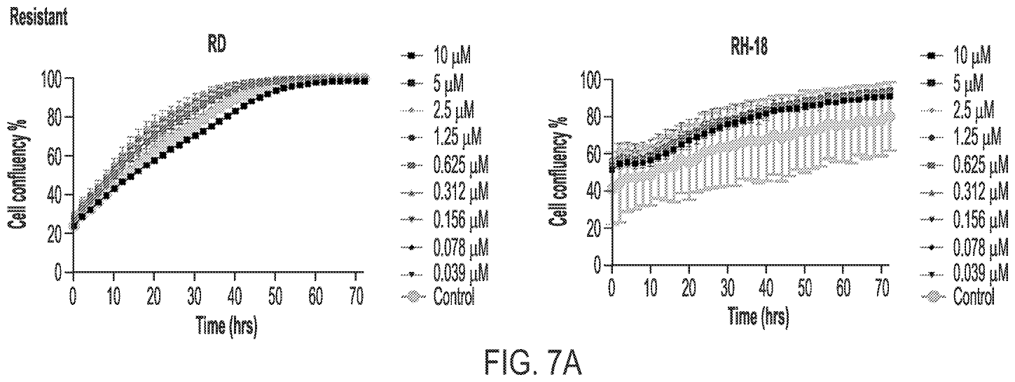

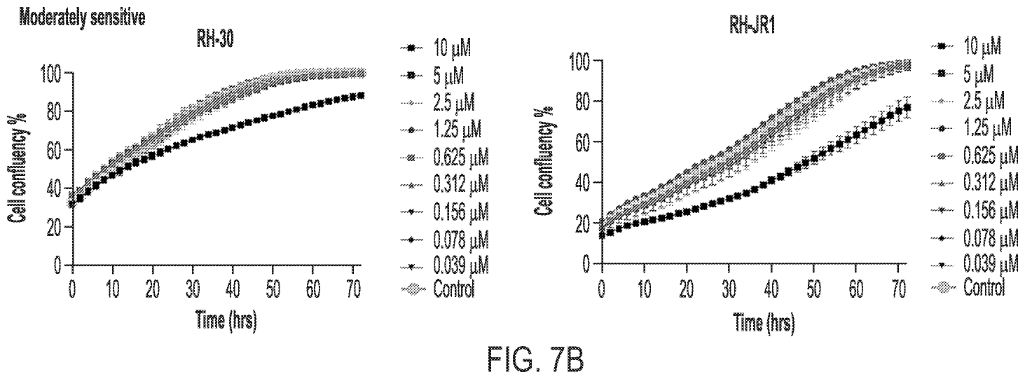

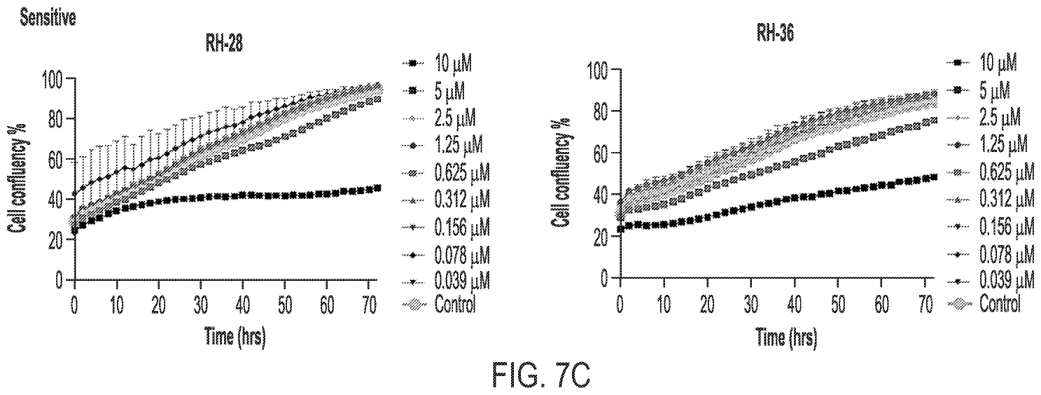

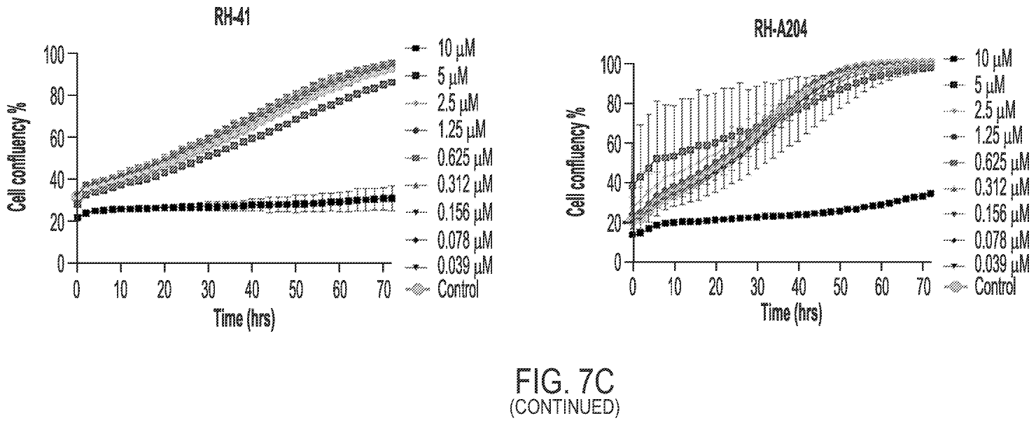

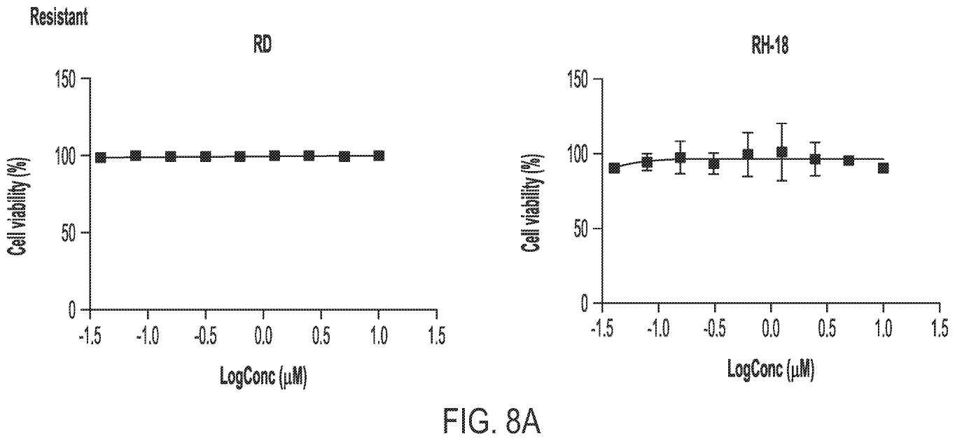

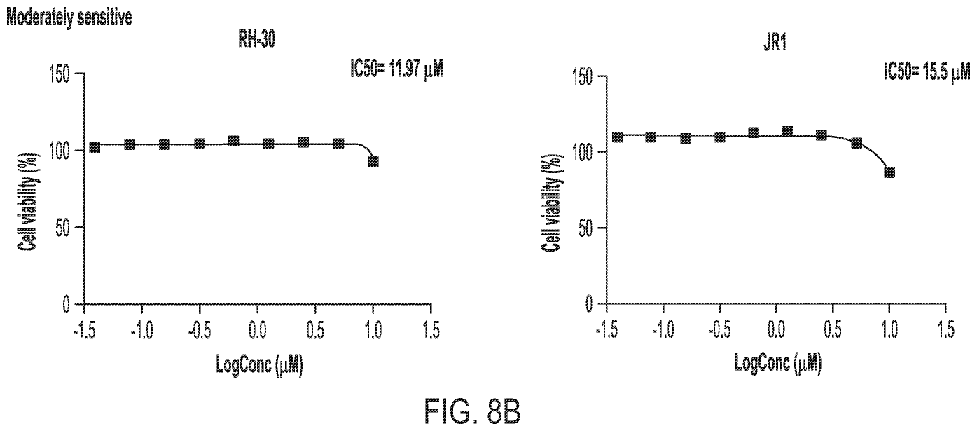

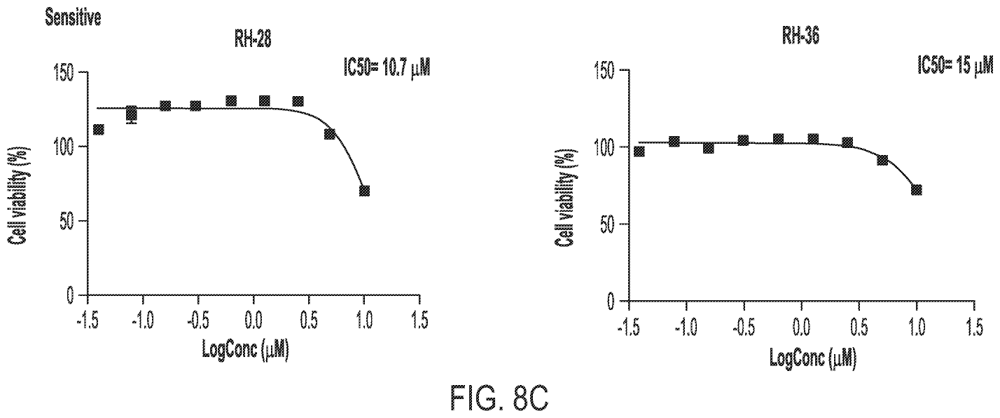

The change in % confluency of Rhabdomyosarcoma cells upon treatment with AT-301 is shown in FIG. 7A-C. The change in % cell viability of Rhabdomyosarcoma cells upon treatment with AT-301 is shown in FIG. 8A-C. Assays were performed as detailed above for FIG. 4A-E.

### 5. AT-301 Attenuates the ATPase Activity of SMARCA2 in Different DNA/Chromatin Substrates

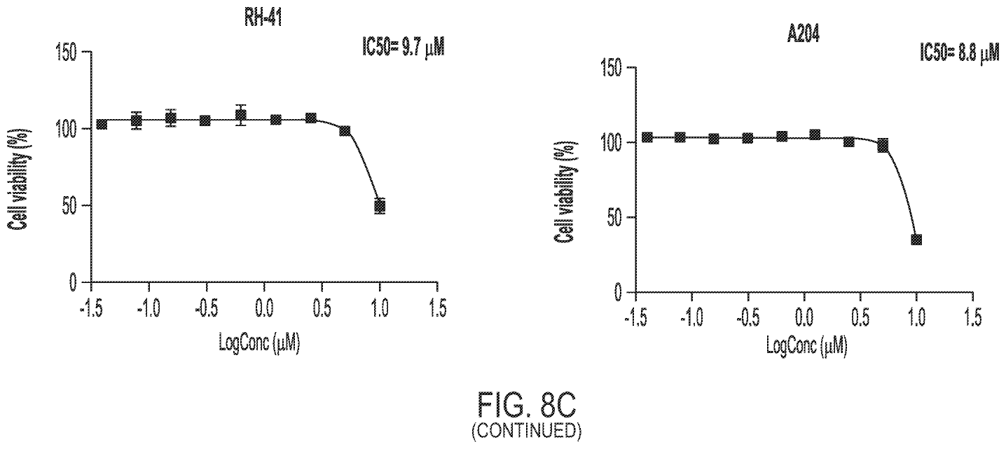

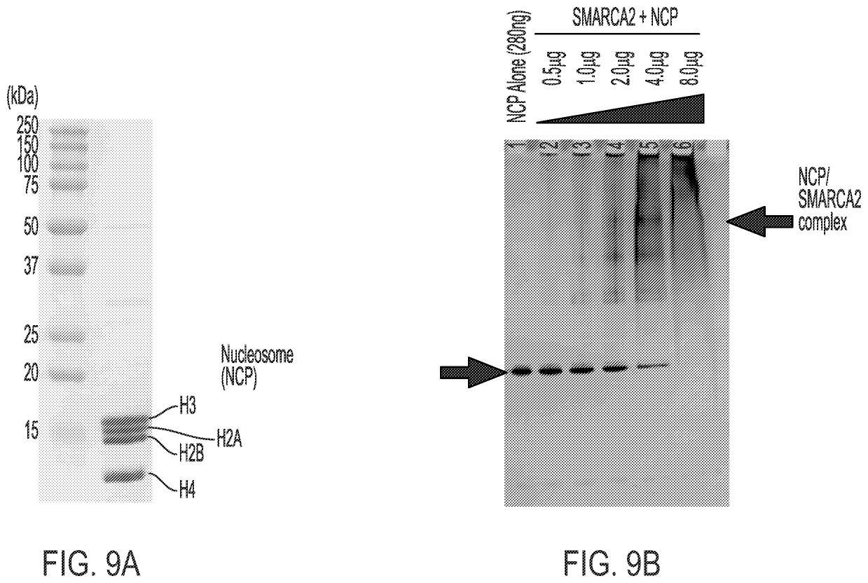

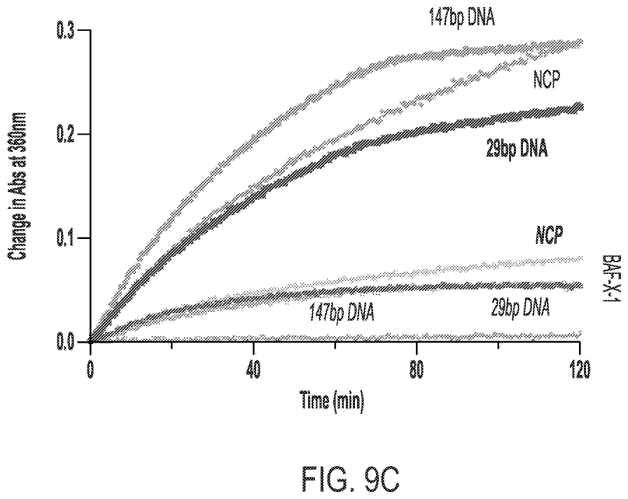

SMARCA2 can interact with nucleosomal, extra nucleosomal DNA, and a DNA wrapped around the histone octamer. These substrates were prepared, and the ATPase activity of SMARCA2 on these substrates were evaluated in the presence and absence of AT-301. Specifically, as shown in FIG. 9A-C, mononucleosome core particles (NCP) were reconstituted from purified histone octamer and 147 bp widom nucleosomal DNA. An electromobility shift assay (EMSA) was used to monitor the direct binding of SMARCA2 to NCP. A clear shift or retarded mobility of NCP/SMARCA2 complex on a native gel suggests direct binding of SMARCA2 to NCP (FIG. 9B). The ATPase activity of SMARCA2 was also evaluated on alternative substrates (a 29-mer duplex DNA, 147 bp nucleosomal DNA, and reconstituted NCP). The same method was used as described in “Purification of Human SMARCA2 Enzyme, DNA Substrate, Enzymatic Assay, and High-Throughput Screen.” AT-301 significantly attenuated the ATPase activity of SMARCA2 assembled on different type of substrates, e.g., short (29-mer duplex DNA), long DNA (147 bp long nucleosomal DNA), and NCP/chromatin, suggesting BAF-X-1's broad potential for inhibition of heterogenous BAF assemblies (residual BAFs) consisting of SMARCA2 that drive tumor growth (FIG. 1C).

### 6. AT-301 Kills a Variety of Different Cancer Cells

a. Cell Lines and Culture Conditions for Cell Growth:

DAOY, D556 cells: EMEM, 10% FBS, Penicillin (100 units/mL) and streptomycin (100 μg/mL) in the presence of 5% CO₂.

Jurkat, Molt-4, RH4, RH30, BT40, GBM2, ES3, ES6, ES7, HC827, H1975, H2030 cells: RPMI 1640, 10% FBS, Penicillin (100 units/mL) and streptomycin (100 μg/mL) in the presence of 5% CO₂.

b. Cell Proliferation Assays and IC₅₀ Measurement:

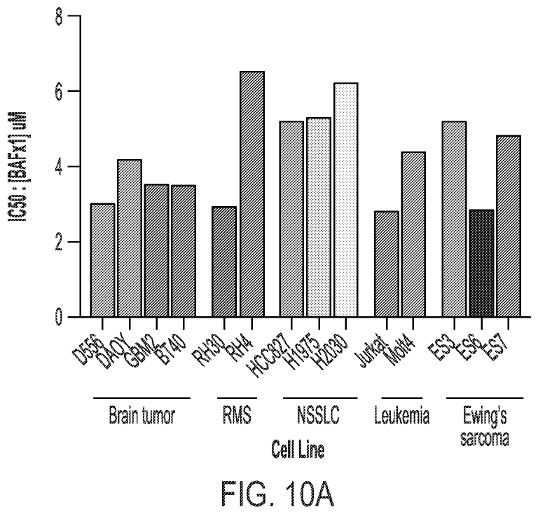

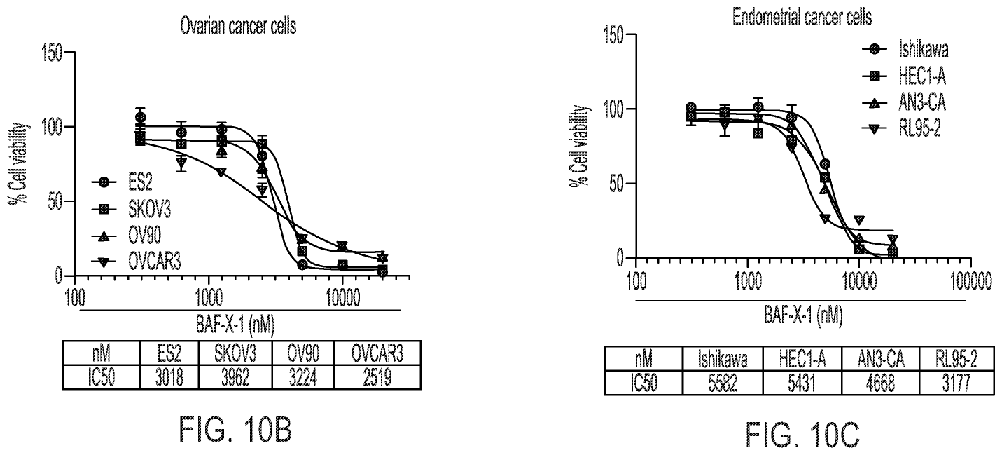

Proliferation of adherent cells was determined on the IncuCyte live-cell imaging system (Essen BioScience). Cells were seeded on clear bottom 96 well plates at a seeding density of 2000 cells/well. Cells were allowed to attach overnight after which the medium was replaced with fresh medium containing serial dilutions (2×) of the compound or DMSO control. Percent confluence was calculated by the IncuCyte software from four different images per well from at least three replicate wells. For non-adherent cells, the 96-well plates were coated with poly-L-lysine before seeding to facilitate cell adhesion. At the end of the assay, dose-dependent cell viability was determined by staining the cells with PrestoBlue (Invitrogen, A13261) as per manufacturer's instructions and IC₅₀ was calculated (FIG. 10).

Referring to FIG. 10A, AT-301 kills a variety of cancer cells (brain tumors, rhabdomyosarcoma, nonsmall cell lung cancer, leukemia, and Ewing's Sarcoma. We also tested whether AT-301 can kill cells representing gynecological cancers (ovarian and endometrial cancer). As shown in FIG. 10B-C, AT-301 kills a variety of ovarian and endometrial cancer cells.

A summary of the anti-cancer activity of AT-301 in various cell lines is shown in Table 3 below.

TABLE 3

| Cell line | IC₅₀ (μM) | R² |
|---|---|---|
| D556 | 3.017 | 0.9763 |
| DAOY | 4.191 | 0.9741 |
| GBM2 | 3.544 | 0.9868 |
| BT40 | 3.492 | 0.9608 |
| RH30 | 2.960 | 0.9778 |
| RH4 | 6.545 | 0.9133 |
| HCC827 | 5.211 | 0.9764 |
| H1975 | 5.306 | 0.9777 |
| H2030 | 6.258 | 0.9979 |
| Jurkat | 2.807 | 0.9073 |
| Molt4 | 4.393 | 0.9504 |
| ES3 | 5.208 | 0.9564 |
| ES6 | 2.840 | 0.8677 |
| ES7 | 4.831 | 0.9717 |

### 7. AT-301 Induces Apoptosis and Alters Cell Cycle

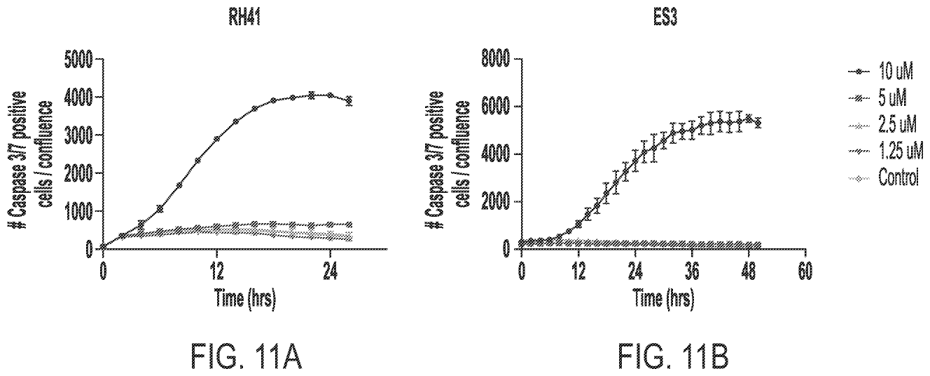

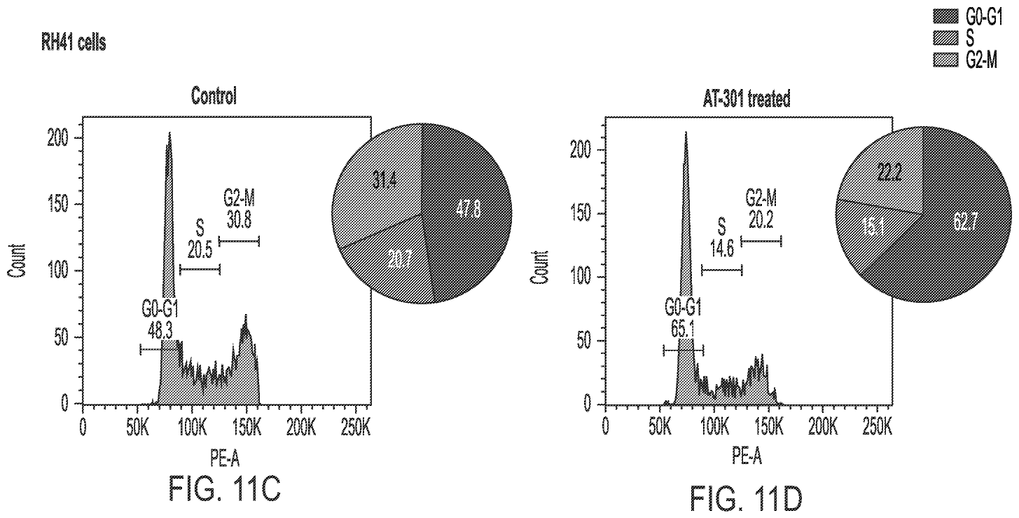

To test whether AT-301 induces apoptosis, RH41 (rhabdomyosarcoma) and ES3 (Ewing's sarcoma) cells were seeded on clear bottom 96-well plates in triplicate at approximately 15,000 cells per well and allowed to adhere for 24-48 hours. The medium was then replaced with fresh medium containing dilutions of the compound (AT-301) or DMSO (as a negative control), and 1× IncuCyte Caspase-3/7 green reagent (Sartorius) and proliferation/apoptosis was measured on IncuCyte live-cell imaging system. The treated cells were imaged every (2/4) hours and four fields from each well were imaged for analysis. As shown in FIG. 11A and FIG. 11B, AT-301 induces apoptosis in these cells.

To test the alterations in cell cycle upon AT-301 treatment, RH41 cells were cultured in 12 well plates, detached using Accutase (Corning, 25-058-Cl), washed with PBS, and fixed in 70% ethanol. For staining, the fixed cells were washed with PBS and stained with FxCycle PI/RNase staining solution (Invitrogen, F10797) as per the supplier's directions. Fluorescence intensity of stained cells was measured on (BD system) and analyzed using FlowJo. As shown in FIG. 11C and FIG. 11D, the majority of RH41 cells treated with AT-301 were arrested in GO-Gi phase of cell cycle.

### 8. Assembly of BAF Complex is Perturbed by AT-301 without Perturbing Oncogenic Fusion Protein Levels

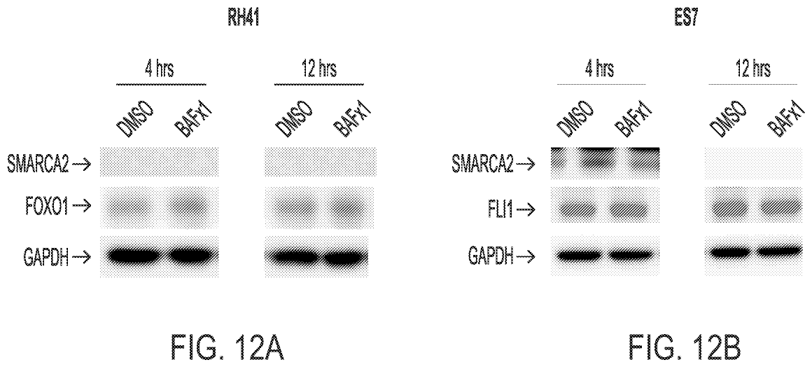

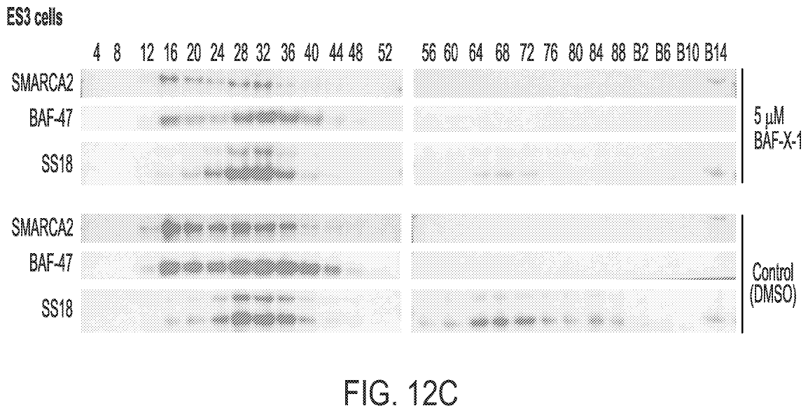

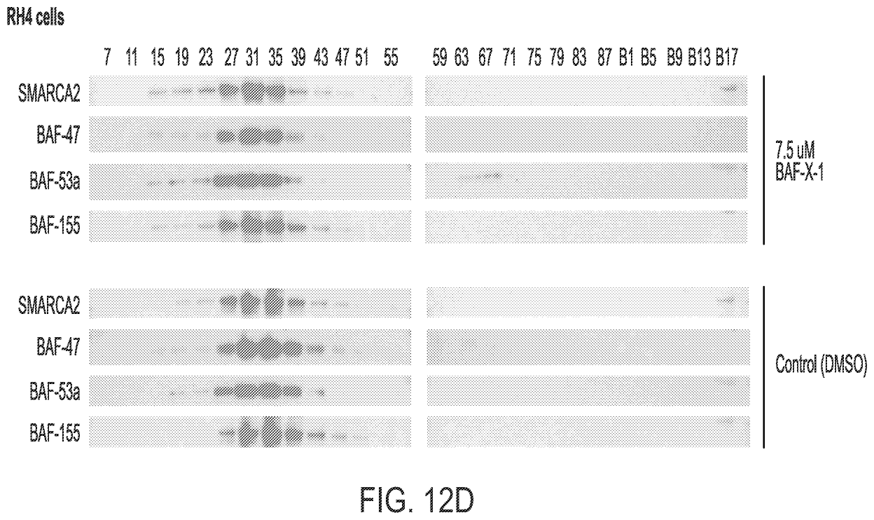

Next, whether the levels of oncogenic transcription factors in RH41 and ES7 cells change upon treatment with AT-301 was tested. Harvested cells were lysed in NP-40 lysis buffer (Thermo Scientific, J60766) supplemented with protease and phosphatase inhibitor (Halt Protease and phosphatase inhibitor cocktail (Thermo Scientific). Total protein was quantified using Bradford assay (Biorad), and 20 μg of it was denatured and separated on pre-cast tris-acetate or tris-glycine gels (Invitrogen). The protein was then transferred to nitrocellulose membranes (Cytiva) and subsequently probed using primary and secondary antibodies. The signal was detected using Clarity and Clarity Max ECL substrates (Biorad) and imaged on Biorad ChemiDoc). As shown in FIG. 12A and FIG. 12B, AT-301 does not alter the expression of the two oncogenic transcription factors in RH41 and ES7 cells.

Next, whether AT-301 can perturb the assembly of BAF complex was evaluated using size-exclusion chromatography (SEC) method. Cells were cultured on 15 cm petri-dishes. Nuclei were extracted and lysed. A HiPrep 16/60 Sephacryl S-400 HR column (GE) was equilibrated with a running buffer and 4 mg of the lysate was loaded on to the column. Fractions of 1 mL were eluted at an elution rate of 0.8 mL/min. Eluted proteins were analyzed by concentrating and immunoblotting every fourth fraction. As shown in FIG. 12C and FIG. 12D, ES3 and RH4 cells treated with 7.5 μM AT-301 showed perturbed assembly of BAF complexes.

### 9. AT-301 Suppresses Tumor Growth of Ewing'S Sarcoma in a Patient Derived Xenograft (PDX) Model

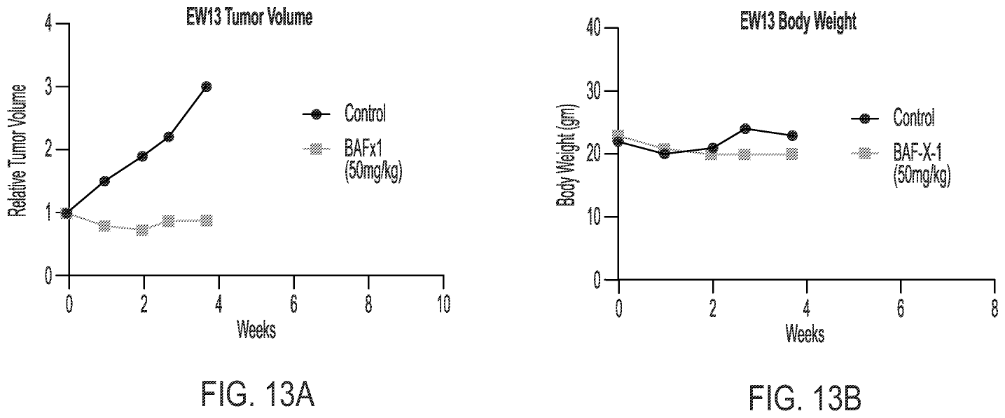

To assess the antitumor activity of AT-301 in a mouse model, a single mouse testing (SMT) experiment was used. SMT has 95% concordance with conventional testing and allows far greater inclusion of genetic/epigenetic heterogeneity. A patient-derived xenograft (PDX) mouse model (C.B.17 scid females (Envigo, IN)) of Ewing's sarcoma (EW13) was used in the SMT experiment. 50 mg/kg of AT-301 (formulated in 30% captisol) or just vehicle (30% captisol) was administered orally three times a week for four weeks. As shown in FIG. 13A and FIG. 13B, AT-301 completely suppressed the tumor growth without any apparent loss in body weight.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 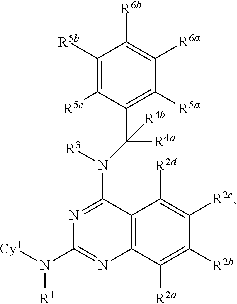
wherein R¹ is selected from hydrogen and C1-C4 alkyl;
wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein R³ is selected from hydrogen and C1-C4 alkyl;
wherein each of R^{4a} and R^{4b} is independently selected from hydrogen, halogen, and C1-C4 alkyl;
wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein R^{6a} is halogen;
wherein R^{6b} is selected from hydrogen and halogen; and
wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl,
provided that either:
(a) R^{6b} is chloro;
(b) R^{6b} is hydrogen; or
(c) R^{6b} is halogen and at least one of R^{5a}, R^{5b}, and R^{5c} is not hydrogen, or a pharmaceutically acceptable salt thereof.

2. The compound of claim 1, wherein R¹ is hydrogen.

3. The compound of claim 1, wherein each of R^{2a}, R^{2b}, R^{2c}, R^{2d}, and R^{2e} is hydrogen.

4. The compound of claim 1, wherein R³ is hydrogen.

5. The compound of claim 1, wherein each of R^{4a} and R^{4b} is hydrogen.

6. The compound of claim 1, wherein each of R^{5a}, R^{5b}, and R^{5c} is hydrogen.

7. The compound of claim 1, wherein R^{6a} is chloro.

8-11. (canceled)

12. The compound of claim 1, wherein Cy¹ is an unsubstituted C3-C6 cycloalkyl.

13. The compound of claim 1, wherein Cy¹ is a cyclopentyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl.

14-15. (canceled)

16. The compound of claim 1, wherein the compound has a structure represented by a formula: 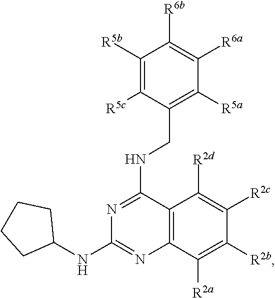
or a pharmaceutically acceptable salt thereof.

17. The compound of claim 1, wherein the compound has a structure represented by a formula: 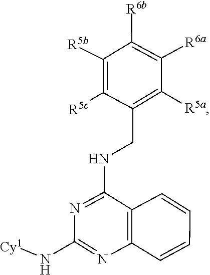
or a pharmaceutically acceptable salt thereof.

18-19. (canceled)

20. The compound of claim 1, wherein the compound is a structure selected from: 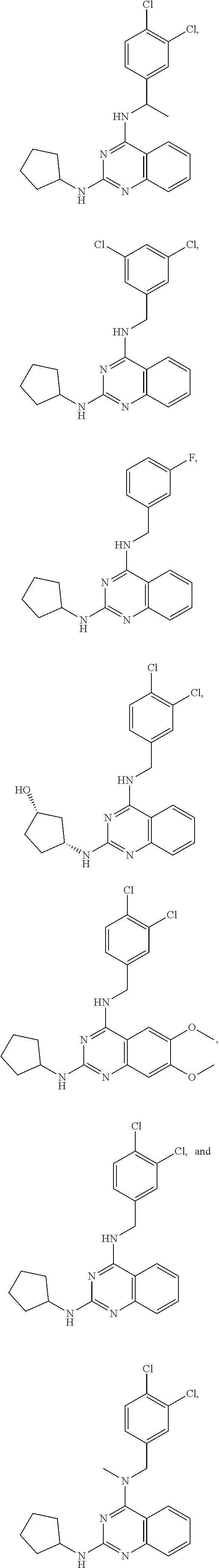
or a pharmaceutically acceptable salt thereof.

21. The compound of claim 1, wherein the compound is: 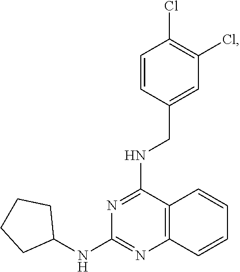 [SMILES: Clc1ccc(CNc2nc(NC3CCCC3)nc3ccccc23)cc1Cl]
or a pharmaceutically acceptable salt thereof.

22. A pharmaceutical composition comprising a therapeutically effective amount of the compound of claim 1, or a pharmaceutically acceptable salt thereof, and a pharmaceutically acceptable carrier.

23-38. (canceled)

39. A method of treating cancer in a subject in need thereof, the method comprising administering to the subject a compound having a structure represented by a formula: 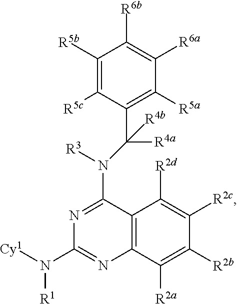
wherein R¹ is selected from hydrogen and C1-C4 alkyl;
wherein each of R^{2a}, R^{2b}, R^{2c}, R^{2d}, and R^{2e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein R³ is selected from hydrogen and C1-C4 alkyl;
wherein each of R^{4a} and R^{4b} is independently selected from hydrogen, halogen, and C1-C4 alkyl;
wherein each of R^{5a}, R^{5b}, and R^{5c} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of R^{6a} and R^{6b} is independently selected from hydrogen and halogen; and
wherein Cy¹ is a C3-C8 cycloalkyl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a} and R^{6b} is halogen, or a pharmaceutically acceptable salt thereof, wherein the cancer comprises a tumor having a mutant BAF chromatin remodeling complex.

40-42. (canceled)

43. The method of claim 39, wherein the compound has a structure represented by a formula: 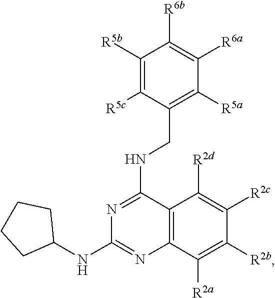
or a pharmaceutically acceptable salt thereof.

44. The method of claim 0, wherein the compound has a structure selected from: 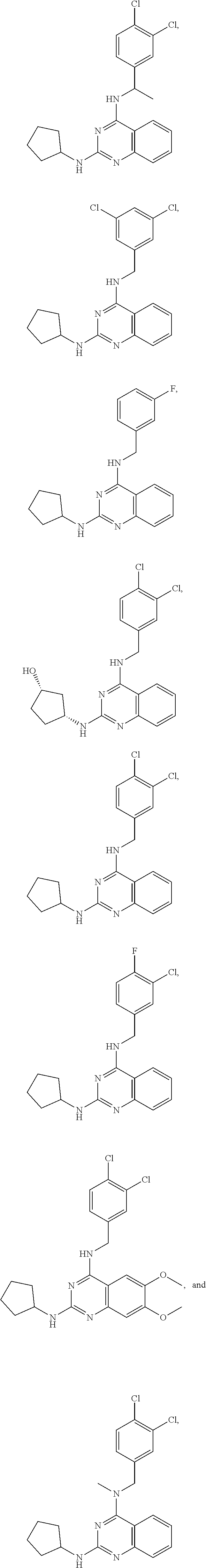
or a pharmaceutically acceptable salt thereof.

45-51. (canceled)

52. The method of claim 39, wherein the cancer is selected from leukemia, neuroblastoma, melanoma, and triple-negative breast cancer.

53. The method of claim 39, wherein the cancer is a pediatric cancer.

54. (canceled)

55. The method of claim 39, wherein the cancer is lung cancer.

56-65. (canceled)


SEQUENCE LISTING (2 sequences)

SEQ ID NO: 1 - DNA, 29 bp, synthetic construct
  source 1..29: mol_type = other DNA
catgcagctgatgcgtacaacacactgtg

SEQ ID NO: 2 - DNA, 29 bp, synthetic construct
  source 1..29: mol_type = other DNA
ttttcccccccccccgcatcagctgcatg